SEQUENCE LISTING (24 sequences)

SEQ ID NO: 1 - DNA, 21 bp, Artificial Sequence
  Description of Artificial Sequence  Synthetic Primer
ggtgttctccatgtcctttgt

SEQ ID NO: 2 - DNA, 21 bp, Artificial Sequence
  Description of Artificial Sequence  Synthetic Primer
ttggggaactgggcagactca

SEQ ID NO: 3 - DNA, 23 bp, Artificial Sequence
  Description of Artificial Sequence  Synthetic Primer
caaacctctggaggaagtgctaa

SEQ ID NO: 4 - DNA, 23 bp, Artificial Sequence
  Description of Artificial Sequence  Synthetic Primer
gtgggaagcacttaattatcaag

SEQ ID NO: 5 - DNA, 23 bp, Artificial Sequence
  Description of Artificial Sequence  Synthetic Primer
ccttgtgcggttgtgttctcatt

SEQ ID NO: 6 - DNA, 23 bp, Artificial Sequence
  Description of Artificial Sequence  Synthetic Primer
tctcacacatccctaggaaccag

SEQ ID NO: 7 - DNA, 23 bp, Artificial Sequence
  Description of Artificial Sequence  Synthetic Primer
tgctgcctccaagaacacaactg

SEQ ID NO: 8 - DNA, 23 bp, Artificial Sequence
  Description of Artificial Sequence  Synthetic Primer
catgatcgtctttagcctttcca

SEQ ID NO: 9 - DNA, 21 bp, Artificial Sequence
  Description of Artificial Sequence  Synthetic Primer
tcgaactctgctgatagccaa

SEQ ID NO: 10 - DNA, 23 bp, Artificial Sequence
  Description of Artificial Sequence  Synthetic Primer
gcagtaaaatgtccttctcctcc

SEQ ID NO: 11 - DNA, 22 bp, Artificial Sequence
  Description of Artificial Sequence  Synthetic Primer
gtgaagcccaaccaacaaagag

SEQ ID NO: 12 - DNA, 22 bp, Artificial Sequence
  Description of Artificial Sequence  Synthetic Primer
ttggaggatgcagctaaagttc

SEQ ID NO: 13 - DNA, 26 bp, Artificial Sequence
  Description of Artificial Sequence  Synthetic Primer
ctgtaatacgactcactatagggaga

SEQ ID NO: 14 - DNA, 27 bp, Artificial Sequence
  Description of Artificial Sequence  Synthetic Primer
ctgatttaggtgacactatagaagagt

SEQ ID NO: 15 - DNA, 61 bp, Artificial Sequence
  Description of Artificial Sequence  Synthetic Primer
gctgatggcgatgaatgaacactgtaatacgactcactatagggagattttttttttttvn

SEQ ID NO: 16 - DNA, 61 bp, Artificial Sequence
  Description of Artificial Sequence  Synthetic Primer
gctgatggcgatgaatgaacactgatttaggtgacactatagaagagttttttttttttvn

SEQ ID NO: 17 - DNA, 168 bp, Artificial Sequence
  Description of Artificial Sequence  Synthetic Primer
taatacgactcactatagggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaaa

SEQ ID NO: 18 - DNA, 24 bp, Artificial Sequence
  Description of Artificial Sequence  Synthetic Primer
gctgatggcgatgaatgaacactg

SEQ ID NO: 19 - DNA, 21 bp, Artificial Sequence
  Description of Artificial Sequence  Synthetic Primer
ggtgttctccatgtcctttgt

SEQ ID NO: 20 - DNA, 23 bp, Artificial Sequence
  Description of Artificial Sequence  Synthetic Primer
caaacctctggaggaagtgctaa

SEQ ID NO: 21 - DNA, 23 bp, Artificial Sequence
  Description of Artificial Sequence  Synthetic Primer
ccttgtgcggttgtgttctcatt

SEQ ID NO: 22 - DNA, 23 bp, Artificial Sequence
  Description of Artificial Sequence  Synthetic Primer
tgctgcctccaagaacacaactg

SEQ ID NO: 23 - DNA, 21 bp, Artificial Sequence
  Description of Artificial Sequence  Synthetic Primer
tcgaactctgctgatagccaa

SEQ ID NO: 24 - DNA, 22 bp, Artificial Sequence
  Description of Artificial Sequence  Synthetic Primer
gtgaagcccaaccaacaaagag

(19) United States
(12) Patent Application Publication
(10) Pub. No.: US 2004/0058373 A1
(43) Pub. Date: Mar. 25, 2004

(54) Competitive amplification of fractionated targets from multiple nucleic acid samples

(76) Inventors: Matthew M. Winkler, Austin, TX (US); David Brown, Austin, TX (US)

Correspondence Address: FULBRIGHT & JAWORSKI L.L.P., 600 CONGRESS AVE., SUITE 2400, AUSTIN, TX, 78701, US

(21) Appl. No.: 10/632,534

(22) Filed: Jul. 31, 2003

Related U.S. Application Data
(63) Continuation of application No. PCT/US02/03169, filed on Jan. 31, 2002.
(60) Provisional application No. 60/265,692, filed on Jan. 31, 2001.
(60) Provisional application No. 60/265,693, filed on Jan. 31, 2001.
(60) Provisional application No. 60/265,695, filed on Jan. 31, 2001.
(60) Provisional application No. 60/265,694, filed on Jan. 31, 2001.

(30) Foreign Application Priority Data
Jan. 31, 2002 (WO) PCT/US02/03168
Jan. 31, 2002 (WO) PCT/US02/02892
Jan. 31, 2002 (WO) PCT/US02/03097
Jan. 31, 2002 (WO) PCT/US02/03169

(51) Int. Cl. C12Q 1/68; C12P 19/34

(52) U.S. Cl. 435/6; 435/91.2

(57) ABSTRACT

Disclosed are methods that allow one or more targets to be compared across two or more nucleic acid populations. The methods rely on first mixing sample populations that are being compared. The sample mixture is then divided into target fractions using hybridization to polynucleotides or oligonucleotides that can be separated from the sample mixture. The target fraction(s) are independently amplified such that the targets from each sample compete for amplification reagents. The amplification products are quantified in a manner that differentiates the sample from which a particular amplification product arose. The relative abundance of amplification products descended from each sample population reflects the level of target present in each of the original samples, providing a direct comparison of the abundance of the target sequences in the samples being characterized.

This patent application claims priority to U.S. Provisional Patent Application No. 60/265,692.

The present application was filed concurrently with: PCT Application No. ______ on Jan. 31, 2002, entitled “COMPARATIVE ANALYSIS OF NUCLEIC ACIDS USING POPULATION TAGGING,” which claims priority to U.S. Provisional Patent Application No. 60/265,694, filed on Jan. 31, 2001, PCT Application No. ______ filed Jan. 31, 2002, entitled “COMPETITIVE POPULATION NORMALIZATION FOR COMPARATIVE ANALYSIS OF NUCLEIC ACID SAMPLES,” which claims priority to U.S. Provisional Patent Application No. 60/265,695 filed on Jan. 31, 2001; and PCT Application No. ______, filed Jan. 31, 2002 entitled “METHODS FOR NUCLEIC ACID FINGERPRINT ANALYSIS,” which claims priority to U.S. Provisional Patent Application No. 60/265,693, filed on Jan. 31, 2001. The disclosure of each of the above-identified applications is specifically incorporated herein by reference in its entirety without disclaimer.

### BACKGROUND OF THE INVENTION

1. Field of the Invention

The present invention relates generally to the fields of nucleic acid amplification. The present invention also relates to methods for adding a nucleic acid tag sequence to a nucleic acid population to promote co-amplification and comparative analysis of multiple nucleic acid samples.

2. Description of Related Art

A recent trend in molecular biology is the study of smaller and smaller tissue samples. Instead of isolating nucleic acids from entire organs or even entire organisms, researchers are now collecting and studying differentiated tissues within organs or even single cells within tissue samples. Smaller samples improve the homogeneity of the nucleic acids being assayed, making it possible to pinpoint genes important in cancer progression, tissue differentiation, and apoptosis. For the most part, nucleic acid amplification must be used to effectively detect and quantify individual RNA and DNA targets within such limited samples.

Competitive polymerase chain reaction (competitive-PCR) is one method for quantifying specific RNA and DNA sequences in samples where only small numbers of target molecules are present. A subtype of competitive-PCR known as competitive reverse transcription polymerase chain reaction (competitive RT-PCR) uses a reverse transcription step to convert an RNA sample to cDNA prior to competitive amplification. In either case, the technique makes use of a synthetic RNA or DNA control, called a competitor, that is co-amplified with an endogenous target using the same amplification reagents in the same reaction. To be quantitative, the competitor and endogenous target must be amplified at the same rate. In addition, the amplification products from the competitor and endogenous target must be distinguishable to allow them to be independently quantified. The latter requirement is typically achieved by making the competitor amplification domain smaller or larger than that of the endogenous target. The competitor is accurately quantified and added at increasing molar concentrations to aliquots of a sample. Competitive-PCR is performed and the amplification products are quantified. Because the amplification rates of the competitor and endogenous target are the same, the ratio of amplification products of the two is equal to the ratio of the competitor and target in the sample that was amplified. Because the concentration of the competitor is known, the concentration of the endogenous target can be determined.

Competitive-PCR and competitive RT-PCR suffer from four drawbacks. First, a competitor must be synthesized, quantified, and tested for each target RNA or DNA being assessed. This requires a substantial outlay of time and effort on the part of the investigator. Second, each sample being assessed is typically aliquoted into multiple reactions with varying quantities of competitor to provide a standard curve against which the nucleic acid target can be accurately quantified. Using multiple reactions to assess each sample is costly both in terms of reagents and time. Where limited samples are being analyzed, this can be a serious limitation. Third, only single targets can be assessed in each set of reactions due to problems with amplifying multiple targets with multiple primers in a single reaction. The second and third drawbacks conspire to limit the number of targets that can be characterized where limited amounts of sample are available. Fourth, only single samples can be assessed in each set of reactions because the endogenous target from one sample cannot be distinguished from the same target in a second sample.

A modification of the competitive RT-PCR procedure reduces the requirement for competitor synthesis and increases the number of samples that can be assessed in a single reaction (Kato 1997, European Patent Application No. 98302726). Adaptor-Tagged Competitive-PCR (ATAC-PCR) makes one sample population a competitor for another sample population. ATAC-PCR accomplishes this by converting mRNA samples to double-stranded cDNA using a reverse transcriptase, digesting the cDNA samples with a restriction enzyme, and ligating adapters to members of the cDNA samples at their respective restriction sites. The adapters for the different samples share a PCR™ primer binding site but are different sizes or possess different restriction sites. The adapter-tagged cDNAs are mixed and amplified with a gene-specific primer and a primer specific to the shared adapter sequence present at the proximal ends of the cDNA populations. If the adapters used for tagging were different sizes, then the products of amplification are directly assessed by gel electrophoresis. If the adapters from the populations differ by a restriction site, then the amplified population must be aliquoted into different restriction digestion reactions to create distinguishable products that can be assessed by gel electrophoresis. Because the amplification products generated from each sample population are different sizes, they can be readily fractionated and quantified. The ratio of amplification products generated from each sample reflects the relative abundance of the target in each sample.

ATAC-PCR has four shortcomings. First, four steps are required to convert an RNA sample to a population that is ready for PCR™ amplification. If any of these steps vary between the samples being compared, inaccuracies will result. Thus inefficient or biased reverse transcription, second strand cDNA synthesis, restriction digestion, or adapter ligation can profoundly affect the data being generated. Second, specificity is dictated by a single primer binding site. The adapter specific primer will bind and extend from any of the cDNAs. Thus if the target specific primer binds a non-target cDNA and primes second strand synthesis, then a non-target cDNA will be exponentially amplified by the action of the tag and target specific primers present in the amplification reaction. Such non-target amplification can affect the amplification of the actual target sequences and thus impact the accuracy of the analysis. Third, ATAC-PCR is apparently limited to the comparative analysis of targets in only a few samples. European Patent Application No. 98302726 and subsequent uses of the technology (Matoba 2000) describe its use to quantify single targets in up to three sample populations. This is apparently due to limitations in resolving more than three amplification products using the size differences possible with ligated adapters. Fourth, only a single target is being assessed in each amplification reaction. This is a burden on both the time required to assess a reasonable number of target sequences and the amount of cDNA sample required to accommodate a reasonable number of amplification reactions.

While the methods described above enable targets to be quantified in small RNA or DNA samples, they are mostly limited to the analysis of only a few target sequences per sample. Methods that facilitate the analysis of more target sequences would be extremely beneficial in the art to determine nucleic acid profiles of limited tissue samples.

### SUMMARY OF THE INVENTION

The present invention overcomes deficiencies in the art by providing methods for first fractionating nucleic acid samples into target fractions and then using nucleic acid amplification to quantitatively assess the nucleic acid target within each fraction. The invention thus allows each target in a sample to be assessed separately without including, and thus wasting, non-target nucleic acids from the sample within the quantitative assay. Fractionating targets within a sample prior to quantitation makes it possible to assess thousands of unique targets in samples that would ordinarily be capable of supporting the assessment of only a few targets.

In particularly preferred embodiments of the invention, nucleic acid tags comprising amplification and differentiation domains are appended to nucleic acids within multiple samples. The differentiation domains for each sample are unique to that sample. Target nucleic acids from different samples may then be fractionated using binding to target specific ligands. Each target fraction may be amplified to provide detectable amounts of the target nucleic acid from the sample mixture. The amplification products are quantified using the unique differentiation domains of sample tags so that it is possible to distinguish from which sample a particular amplification product arose.

The present invention encompasses methods of comparing one or more nucleic acid targets within two or more samples. In a general embodiment, these methods comprise:

a) preparing a sample mixture by a process comprising obtaining at least a first sample and a second sample, each potentially having at least a first nucleic acid target and mixing the first nucleic acid sample and the second nucleic acid sample to create a sample mixture;

b) isolating at least a first target fraction of the sample mixture;

c) performing at least a first amplification reaction on the first target fraction, wherein the amplification reaction produces at least a first amplified nucleic acid, if the first nucleic acid target is present in the first sample, and at least a second amplified nucleic acid, if the first nucleic acid target is present in the second sample;

d) differentiating the first amplified nucleic acid in the first target fraction, if any, from the second amplified nucleic acid in the first target fraction, if any; and

e) comparing abundance of the first nucleic acid target of said first sample to abundance of the first nucleic acid target of said second sample.

In is not necessary that the first target nucleic acid be present in either the first sample or the second sample. Rather, the practice of the methods disclosed herein is appropriate to determine whether or not the first target nucleic acid, or any other target nucleic acid, is present in a given sample. Comparing abundance of a given target in given samples is possible, even if the target is absent from one or more of the samples. Of course, in many embodiments, the first nucleic acid target is present in the first sample and/or the second sample.

While, at its most basic level, there can be only one nucleic acid of interest in the samples, the advantages of the invention allow one to analyze a variety of nucleic acid targets in the samples. The advantages of the invention are especially relevant where the samples being compared comprise very small amounts of nucleic acid (for instance, less the 1 μg of total RNA, genomic DNA, or both. Therefore, in many instances, the first nucleic acid target will be only one of a plurality of nucleic acid targets to be analyzed in the samples. Further, while, at the most basic level, the methods of the invention may be employed with only two samples, in many cases, the first and second sample are two samples of a plurality of samples. Also, in many cases, the first target fraction is one of a plurality of target fractions.

In one embodiment of the invention, at least a first amplification reaction is performed on the first target fraction using at least a first target-specific primer, wherein the amplification reaction produces at least a first amplified nucleic acid, if the first nucleic acid target is present in the first sample, and at least a second amplified nucleic acid, if the first nucleic acid target is present in the second sample. An even more specific embodiment involves isolating at least a second target fraction from the sample mixture and performing a second amplification reaction on the second target fraction using a second target-specific primer that is specific for a second target that may be present in the first sample and/or the second sample.

The nucleic acid target can be an RNA, DNA or a combination thereof. It is not required that the nucleic acid target be of natural origin, and the target can contain synthetic nucleotides. In specific aspects, the nucleic acid target is an RNA, for example, prokaryotic or eukaryotic RNA, total RNA, polyA RNA, an in vitro RNA transcript or a combination thereof. In other facets, the nucleic acid target may comprise DNA, such as, for example, cDNA, genomic DNA or a combination thereof. In certain aspects, at least one of the samples comprises nucleic acid isolated from a biological sample from, for example, a cell, tissue, organ or organism. In other aspects, at least one of the samples may comprise nucleic acid from an environmental sample. Of course, there is no need for all of the samples compared in a particular assay to be of the same source or type of source. A single sample may contain nucleic acid from a single source, or it may be the result of combining nucleic acids from multiple sources.

The above portion of the summary relates to some generic embodiments of the invention in which samples may, but need not be tagged. The following paragraphs describe primarily some preferred embodiments using samples that are tagged.

In preferred embodiments of the invention, preparing the sample mixture is further defined as comprising: preparing at least a first tagged nucleic acid sample by appending at least a first nucleic acid tag comprising a first amplification domain and a first differentiation domain to the first nucleic acid target of the first sample, if the first nucleic acid target is present in the first sample; preparing at least a second tagged nucleic acid sample by appending at least a second nucleic acid tag comprising a second amplification domain and a second differentiation domain to the first nucleic acid target of the second sample, if the first nucleic acid target is present in the second sample, wherein the second differentiation domain is different from the first differentiation domain; and mixing the first tagged nucleic acid sample and the second tagged nucleic acid sample to create the sample mixture. In this embodiment, performing at least a first amplification reaction on the first target fraction is further defined as producing at least a first amplified nucleic acid comprising the first differentiation domain and a segment of the first nucleic acid target, if the first nucleic acid target is present in the first sample, and at least a second amplified nucleic acid comprising the second differentiation domain and a segment of the first nucleic acid target, if the first nucleic acid target is present in the second sample.

In many applications, the nucleic acid target and/or the nucleic acid tag will be single-stranded nucleic acid. However this in not required in all embodiments of the invention, and those of skill will be able to follow the teachings of the specification to employ double-stranded nucleic acids in the invention.

As discussed above, one of the advantages of the invention is the ability of it to be used to analyze many samples simultaneously. In preferred embodiments, the tags used for each sample will comprise a differentiation domain that is unique to that sample. Of course, in cases where there are a plurality of samples, there will typically be a plurality of tags. Those of skill in the art will be able to employ the teachings of this specification to prepare appropriate tags. Typically, the number of unique tags required for a given procedure will be equal to the number of samples to be analyzed. The nucleic acid target can be one of a plurality of nucleic acid targets within the samples. Likewise, the first and second samples are two samples of a plurality of samples, and the first and second tag are two tags of a plurality of tags.

In presently preferred embodiments of the invention, the differentiation domains of the tags are appended between the nucleic acid target sequence and the amplification domain. In this manner, the differentiation domain is assured of being amplified during the amplification process, and is present in the amplified nucleic acid. Of course, those of skill in the art will realize that there are other positions of the differentiation and amplification domains in tags, and will be able to utilize tags with the domains in a variety of functional positions.

The amplification domains of nucleic acid tags may comprise any appropriate sequences as described elsewhere in the specification or known to those of skill in the art. In some preferred embodiments, the amplification domain comprises a primer binding domain and/or a transcription domain. In many cases, the amplification domains are the same for all targets being assessed within a sample. However, in some embodiments the amplification domains could be specific for a nucleic acid target.

In preferred embodiments, the amplification domain for a first nucleic acid sample will be functionally equivalent to the amplification domain of a second sample and functionally equivalent to any amplification domains of any other samples. As used in this manner, “functionally equivalent” means that the amplification domains provide amplification of the target nucleic acid in the same manner and at the same rate. In the simplest embodiments of the invention, the amplification domain for a first nucleic acid target of a first sample will be identical to the amplification domains of the same target in any other samples.

The differentiation domains useful in the invention can be of any form described elsewhere in this specification or apparent to those of skill in view of the specification. In preferred embodiments, the differentiation domain will comprise at least a size differentiation domain, an affinity domain, or a unique sequence domain.

In preferred embodiments, target fractions within a sample mixture are isolated by binding target-specific ligands to at least a segment of the nucleic acid targets. Ligands can be nucleic acids, proteins, or other biomolecules that will bind to the targets. In a specific embodiment, a target-specific ligand is a nucleic acid complementary to at least a segment of a first nucleic acid target. In such cases, the first complementary nucleic acid may be used to separate the first target nucleic acid from at least one other nucleic acid or molecule. The target fraction may be subsequently removed from the first complementary nucleic acid and assessed in one of a variety of methods employing nucleic acid amplification. In many cases, the first complementary nucleic acid is one of a plurality of complementary nucleic acids, and each complementary nucleic acid is complementary to one of a plurality of nucleic acid targets. The first complementary nucleic acid may be bound to a solid support. For example, the first complementary nucleic acid can be one of a plurality of complementary acids bound to an array, and each of the complementary nucleic acids can be complementary to one of a plurality of nucleic acid targets. The solid support can be one of a plurality of solid supports. For example, the solid support can be an array, a microtiter well, a chip, a bead or a combination thereof.

In many embodiments, differentiating comprises binding the first amplified nucleic acid to a ligand specific to at least a segment of the first differentiation domain or binding the second amplified nucleic acid to at least a segment of the second differentiation domain. In some cases, the differentiation domain of the first nucleic acid tag comprises a first affinity domain and the second nucleic acid tag comprises a second affinity domain that is distinct from the first affinity domain. In specific such cases, the differentiating comprises binding at least a segment of the first affinity domain to a first affinity domain specific ligand and/or binding at least a segment of the second affinity domain to a second affinity domain specific ligand. The first and second affinity domain specific ligands can be two of a plurality of ligands. Further, at least one of the first or the second affinity domain specific ligands may be bound to at least one solid support, which one solid support can be one of a plurality of solid supports such as, for example, an array, a microtiter well, a glass surface, a chip, a bead or a combination thereof.

In preferred embodiments, amplification products from a given target fraction are labeled and the labeled nucleic acids are hybridized to unlabeled ligands bound to an array. The differentiated labeled amplification products are then quantified. In some embodiments, at least one of the first or the second affinity domain specific ligands is labeled. Such labeling can be performed in a manner such that the binding of the first affinity domain specific ligand to the first affinity domain results in a detectable signal and/or the binding of the second affinity domain specific ligand to the second affinity domain results in a detectable signal. In some preferred embodiments, the binding of the first affinity domain specific ligand to the first affinity domain results in a first detectable signal and the binding of the second affinity domain specific ligand to the second affinity domain results in a second detectable signal. Typically, the first detectable signal is distinguishable from the second detectable signal.

In other embodiments, the differentiation domain of the first nucleic acid tag comprises a first sequence domain and the differentiation domain of the second nucleic acid tag comprises a second sequence domain that is distinguishable from the first sequence domain. In such cases, differentiating can comprise sequencing the first amplified nucleic acid and the second amplified nucleic acid. In such cases, the amplified nucleic acid may be cloned prior to sequencing.

In other embodiments the differentiation domain is a unique size domain, and differentiation may comprise distinguishing amplified products by size.

The first nucleic acid tag or the second nucleic acid tag may further comprise at least one additional domain. For example, the additional domain can be a restriction enzyme domain, a secondary amplification domain, a sequencing primer binding site, a labeling domain or a combination thereof. In cases where the additional domain comprises a labeling domain, the labeling domain can be a transcription promoter or a primer binding site.

A preferred method of the invention comprises:

a) obtaining at least a first sample and a second sample, each potentially having at least a first nucleic acid target;

b) preparing at least a first tagged nucleic acid sample by appending at least a first nucleic acid tag comprising a first amplification domain and a first differentiation domain to the first nucleic acid target of the first sample, if the first nucleic acid target is present in the first sample;

c) preparing at least a second tagged nucleic acid sample by appending at least a second nucleic acid tag comprising a second amplification domain and a second differentiation domain to the first nucleic acid target of the second sample, if the first nucleic acid target is present in the second sample;

d) mixing the first tagged nucleic acid sample and the second tagged nucleic acid sample to create a sample mixture;

e) isolating at least a first target fraction of the sample mixture;

f) performing at least a first amplification reaction on the first target fraction, wherein the amplification reaction produces at least a first amplified nucleic acid comprising the first differentiation domain and a segment of the first nucleic acid target, if the first nucleic acid target is present in the first sample, and at least a second amplified nucleic acid comprising the second differentiation domain and a segment of the first nucleic acid target, if the first nucleic acid target is present in the second sample;

g) differentiating the first amplified nucleic acid in the first target fraction, if any, from the second amplified nucleic acid in the first target fraction, if any; and

h) comparing the first nucleic acid target of said first sample to the nucleic acid target of said second sample.

While a more specific method of the invention comprises:

a) obtaining at least a first sample and a second sample, each potentially having at least a first nucleic acid target;

b) preparing at least a first tagged nucleic acid sample by appending at least a first nucleic acid tag comprising a first amplification domain and a first differentiation domain to the first nucleic acid target of the first sample, if the first nucleic acid target is present in the first sample, wherein the first differentiation domain comprises a first affinity domain;

c) preparing at least a second tagged nucleic acid sample by appending at least a second nucleic acid tag comprising a second amplification domain and a second differentiation domain to the first nucleic acid target of the second sample, if the first nucleic acid target is present in the second sample, wherein the first differentiation domain comprises a second affinity domain that is distinct from the first differentiation domain;

d) mixing the first tagged nucleic acid sample and the second tagged nucleic acid sample to create a sample mixture;

e) isolating at least a first target fraction of the sample mixture;

f) performing at least a first amplification reaction on the first target fraction, wherein the amplification reaction produces at least a first amplified nucleic acid comprising the first affinity domain and a segment of the first nucleic acid target, if the first nucleic acid target is present in the first sample, and at least a second amplified nucleic acid comprising the second affinity domain and a segment of the first nucleic acid target, if the first nucleic acid target is present in the second sample;

g) differentiating the first amplified nucleic acid in the first target fraction, if any, from the second amplified nucleic acid in the first target fraction, if any, by binding the first affinity domain to a first ligand and the second affinity domain to a second ligand; and

h) comparing the first nucleic acid target of said first sample to the nucleic acid target of said second sample. The method of the present invention incorporates competitive nucleic acid amplification as do other procedures, but it differs in that the sample being amplified is fractioned prior to amplification. This allows us to quantify many more target sequences in a given sample because we are not wasting non-target sequences in the various amplification reactions.

The present invention differs from ATAC-PCR in several manners. In preferred embodiments of the present invention, a nucleic acid population being amplified is single-stranded and uses the action of a target specific primer to initiate amplification. In contrast, ATAC-PCR initiates amplification with double-stranded nucleic acids that all possess a domain that is complementary to the adapter-specific primer. Therefore, target and non-target sequences are at least linearly amplified from the amplification domain of the adapter. This generates background that is not found using the single-stranded material that initiates amplification in preferred embodiments of the present invention.

In certain embodiments of the present invention, analysis of differentiated populations does not rely upon differences in target(s) size. Thus, the methods of the present invention may analyze or compare a virtually unlimited number of samples. In contrast, ATAC-PCR suffers functional limitations due to its reliance upon size to differentiate targets amplified from different samples.

As used herein in the specification, “a” or “an” may mean one or more. As used herein in the claim(s), when used in conjunction with the word “comprising”, the words “a” or “an” may mean one or more than one. As used herein “another” may mean at least a second or more. As used herein, a “plurality” means “two or more.”

As used herein, “plurality” means more than one. In certain specific aspects, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 400, 500, 750, 1,000, 2,000, 3,000, 4,000, 5,000, 7,500, 10,000, 15,000, 20,000, 30,000, 40,000, 50,000, 60,000, 70,000, 80,000, 90,000, 100,000, 125,000, 150,000, 200,000 or more, and any integer derivable therein, and any range derivable therein.

As used herein, “any integer derivable therein” means a integer between the numbers described in the specification, and “any range derivable therein” means any range selected from such numbers or integers.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic for Fractionation/Competitive Amplification of Tagged Samples.

FIG. 2. Tagged Nucleic Acid Targets.

### DESCRIPTION OF ILLUSTRATIVE EMBODIMENTS

A primary application of the invention is quantitative expression analysis of nucleic acids. The competitive-PCR™ methods disclosed herein allow many different nucleic acid targets to be quantified in minimal amounts of sample. In addition, these methods allow many samples to be characterized simultaneously to reduce the time and material costs associated with nucleic acid analysis.

In specific embodiments, these methods involve mixing a sample RNA or DNA with a collection of target-specific competitors. Targets within the mixture are fractionated into discrete sub-populations. The fractions are then assayed using target specific competitive amplification to determine the relative abundance of each target in the sample populations. Because the samples are divided into specific target fractions prior to amplification, even limited nucleic acid samples can be used to characterize many different targets. The methods of the present invention can be relatively quantitative or absolutely quantitative depending on whether the concentrations of the target-specific competitors are known.

In a preferred embodiment of the invention, nucleic acid tags are appended to the nucleic acids comprising two or more samples. In preferred aspects, the tag sequences are different for each of the nucleic acid samples being analyzed. In certain embodiments, the differentially tagged nucleic acids may be mixed and bound to an agent (e.g., hybridized to nucleic acid) that may be used to isolate target sequences within the sample mixture away from the other nucleic acids in the sample mixture.

In certain preferred embodiments, each target fraction may then be amplified using at least one primer specific to the target and at least one primer specific to an amplification domain present in the appended tags. The amplified nucleic acid(s) may be quantified using one of several methods, described herein or that would be known to one of skill in the art in light of the present disclosures, that can distinguish the sample-specific tag sequences present in each collection of amplified nucleic acid. Because the target nucleic acids from the different samples are co-amplified, the relative abundance of the sample specific tag sequence in the amplified population will reflect the relative abundance of the target in the original nucleic acid samples.

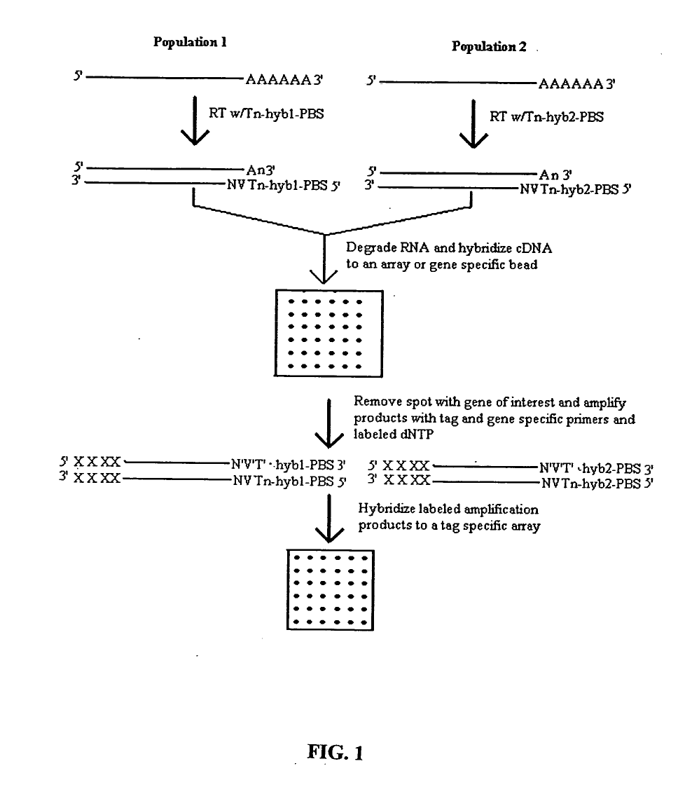

FIG. 1 illustrates one possible method for assaying samples using a preferred embodiment of the invention, which compares one or more RNA targets within two or more nucleic acid populations. For clarity, in FIG. 1, only two nucleic acid populations are pictured; in reality there are likely to be a plurality of nucleic acid populations used per analysis. In many embodiments, the first RNA target is one of a plurality of nucleic acid targets (e.g., 2 to 1100,000 different nucleic acid targets).

As shown in FIG. 1, a first nucleic acid tag comprising an amplification domain (i.e., a primer binding domain, PBS) and a differentiation domain comprising a first affinity domain (i.e., hyb 1) is appended (via reverse transcription) to a first RNA target of a first nucleic acid population. A second nucleic acid tag comprising an amplification domain (i.e., a primer binding domain, PBS) and a differentiation domain comprising a second affinity domain (i.e., hyb 2) is appended to the first RNA target of a second nucleic acid population. The differentiation domain of the second nucleic acid tag is different than the differentiation domain of the first nucleic acid tag. The amplification domains are typically identical, or, at the least, functionally equivalent.

As shown in FIG. 1, the tagged cDNA samples are mixed to form a sample mixture. The sample mixture is then fractionated to form one or more target fractions.

The nucleic acid targets can be fractionated by annealing to one or more target or tag specific ligand(s). Such ligands can be bound to a solid support (e.g., an array or bead).

Once a target fraction comprising nucleic acid targets has been separated from other nucleic acids in the sample mixture, it may be annealed to and amplified (e.g., via PCR™) with one or more primers specific to target and tag sequences producing a first amplified nucleic acid comprising the differentiation domain of the first nucleic acid tag and a nucleic acid segment of the target nucleic acids from the first sample, and a second amplified nucleic acid comprising the differentiation domain of the second nucleic acid tag and a nucleic acid segment of the target nucleic acids from the second sample. These amplified nucleic acids can be labeled, as taught elsewhere in the specification.

The amplified nucleic acids are differentiated and the differentiated nucleic acids compared to determine the abundance (i.e., concentration) of the first nucleic acid target in the first population relative to the abundance of that target in the second population. In a preferred embodiment of the invention, differentiation may be achieved by binding to tag specific ligands (e.g., ligands specific for the different differentiation domains). The tag-specific ligands may be bound to a solid support such as, for example, an array.

One particularly preferred embodiment of the invention involves the use of two arrays for analysis. In this case, the tagged cDNA targets are hybridized to appropriate probes on a first array, after which an individual address on the array can be excised to provide a relatively pure sample of cDNA possessing sequence complementary to the probe. The hybridized cDNA is removed from the array support and amplified with primers specific to the tag and target. The amplification reaction incorporates a detectable moiety to create labeled nucleic acid(s). The amplified nucleic acid(s) are then denatured and hybridized to a second array that has oligonucleotides specific to each of the different tag sequences attached at known addresses. The signal from each address is quantified, providing the relative abundance of the target sequence in each of the original nucleic acid samples.

A. Nucleic Acids: Tags and Samples

Embodiments of the present invention involve nucleic acids in many forms. Nucleic acid samples are collections of RNA and/or DNA derived or extracted from chemical or enzymatic reactions, biological samples, or environmental samples. Nucleic acid tags are nucleic acids of a defined sequence that are appended to nucleic acids in a sample to facilitate its analysis. There are many potential types of tags for use in the invention, which are described elsewhere in this specification.

1. General Description of Nucleic Acids

The general term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” The term “oligonucleotide” refers to a molecule of between 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleobases in length, and any range derivable therein. The term “polynucleotide” refers to at least one molecule of greater than about 100 nucleobases in length.

a. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table of non-limiting purine and pyrimidine derivatives and analogs is also provided herein below.

TABLE 1
Purine and Pyrmidine Derivatives or Analogs

| Abbr. | Modified base description |
|---|---|
| Ac4c | 4-acetylcytidine |
| Chm5u | 5-(carboxyhydroxylmethyl) uridine |
| Cm | 2′-O-methylcytidine |
| Cmnm5s2u | 5-carboxymethylamino-methyl-2-thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| M1a | 1-methyladenosine |
| M1f | 1-methylpseudouridine |
| M1g | 1-methylguanosine |
| M1I | 1-methylinosine |
| M22g | 2,2-dimethylguanosine |
| M2a | 2-methyladenosine |
| M2g | 2-methylguanosine |
| M3c | 3-methylcytidine |
| M5c | 5-methylcytidine |
| M6a | N6-methyladenosine |
| M7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2-methylthiopurine-6- |
| | yl)carbamoyl)threonine |
| Mt6a | N-((9-beta-D-ribofuranosylpurine-6-yl)N-methyl- |
| | carbamoyl)threonine |
| Mv | Uridine-5-oxyacetic acid methylester |
| O5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

A nucleobase may be comprised in a nucleoside or nucleotide, using any chemical or natural synthesis method described herein or known to one of ordinary skill in the art.

b. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Komberg and Baker, 1992).

c. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

d. Nucleic Acid Analogs

A tag or other nucleic acid used in the invention may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability; U.S. Pat. No. 5,223,618 which describes oligonucleotide analogs with a 2 or 3 carbon backbone linkage attaching the 4′ position and 3′ position of adjacent 5-carbon sugar moiety to enhanced resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance; U.S. Pat. No. 5,214,136 which describes olignucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance and binding affinity; and U.S. Pat. No. 5,708,154 which describes RNA linked to a DNA to form a DNA-RNA hybrid.

e. Polyether and Peptide Nucleic Acids

In certain embodiments, it is contemplated that a tag or other nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et. al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art, and are encompassed by the invention. In a non-limiting example, U.S. Pat. No. 5,786,461 describes PNAs with amino acid side chains attached to the PNA backbone to enhance solubility of the molecule. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

f. Preparation of Nucleic Acids

A tag or other nucleic acid used in the invention may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides are used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid includes one produced by enzymes in amplification reactions such as PCR (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

g. Nucleic Acid Purification

A tag or other nucleic acid used in the invention may be purified on polyacrylamide gels, cesium chloride centrifugation gradients, or by any other means known to one of ordinary skill in the art (see for example, Sambrook et al. 1989, incorporated herein by reference).

In particular embodiments, tags or other nucleic acid used in the invention may be isolated from at least one organelle, cell, tissue or organism. In certain embodiments, “isolated nucleic acid” refers to a nucleic acid that has been isolated free of, or is otherwise free of, the bulk of cellular components such as for example, macromolecules such as lipids or proteins, small biological molecules, and the like.

h. Nucleic Acid Complements

The present invention also encompasses a nucleic acid that is complementary to a specific nucleic acid sequence. A nucleic acid “complement(s)” or is “complementary” to another nucleic acid when it is capable of base-pairing with another nucleic acid according to the standard Watson-Crick, Hoogsteen or reverse Hoogsteen binding complementarily rules. As used herein “another nucleic acid” may refer to a separate molecule or a spatial separated sequence of the same molecule.

i. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suit a particular application.

B. Nucleic Acid Samples (Populations)

The invention can be applied to the comparative analysis of any nucleic acid population. The nucleic acids can be RNA, DNA, or both. The nucleic acids can be part of a collection of other molecules, including proteins, carbohydrates or small molecules. While the population can comprise even a single sequence, the method is best suited for nucleic acid samples that include hundreds or thousands of unique sequences.

The terms “target”, “target nucleic acid” and “target sequence” refer to one or more nucleic acids (e.g., DNA, RNA) of a specific sequence that are being characterized. Often, target nucleic acids comprise a sub-population of nucleic acids relative to all the nucleic acid sequences originally present in a nucleic acid sample.

1. Sources of Nucleic Acid Samples

Nucleic acid samples can be obtained from biological material, such as cells, tissues, organs or organisms. The invention is particularly relevant to total and polyA RNA preparations from tissues or cells. Similarly, the invention could be applied to cDNAs derived from cells or tissues. In other embodiments, multiple genomic DNA samples could be assessed using the methods of the present invention.

a. Cells and Tissues

A cell, or a tissue comprising cells, may be a source of nucleic acids for the present invention. In certain embodiments, cells or tissue may be part or separated from an organism. In certain embodiments, a cell or tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endotbelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, anthers, ascites, cobs, ears, flowers, husks, kernels, leaves, meristematic cells, pollen, root tips, roots, silk, stalks, and all cancers thereof.

b. Organisms

In certain embodiments, the cell or tissue may be comprised in at least one organism. In certain embodiments, the organism may be, but is not limited to, a eubacteria, an archaea, a eukaryote or a virus (for example, webpage http://phylogeny.arizona.edu/tree/phylogeny.html).

i. Eubacteria

In certain embodiments, the organism is a eubacteria. In particular embodiments, the eubacteria may be, but is not limited to, an aquifecales; a thermotogales; a thermodesulfobacterium; a member of the thermus-deinococcus group; a chloroflecales; a cyanobacteria; a finnicutes; a member of the leptospirillum group; a synergistes; a member of the chlorobium-flavobacteria group; a member of the chlamydia-verrucomicrobia group, including but not limited to a verrucomicrobia or a chlamydia; a planctomycetales; a flexistipes; a member of the fibrobacter group; a spirochetes; a proteobacteria, including but not limited to an alpha proteobacteria, a beta proteobacteria, a delta & epsilon proteobacteria or a gamma proteobacteria. In certain aspects, an organelle derived from eubacteria are contemplated, including a mitochondria or a chloroplast.

ii. Archaea

In certain embodiments, the organism is an archaea (a.k.a. archaebacteria; e.g., a methanogens, a halophiles, a sulfolobus). In particular embodiments, the archaea may be, but is not limited to, a korarchaeota; a crenarchaeota, including but not limited to, a thermofilum, a pyrobaculum, a thermoproteus, a sulfolobus, a metallosphaera, an acidianus, a thermodiscus, a igneococcus, a thermosphaera, a desulfurococcus, a staphylothermus, a pyrolobus, a hyperthermus or a pyrodictium; or an euryarchaeota, including but not limited to a halobacteriales, methanomicrobiales, a methanobacteriales, a methanococcales, a methanopyrales, an archeoglobales, a thermoplasmales or a thermococcales.

iii. Eukaryotes

In certain embodiments, the organism is a eukaryote (e.g., a protist, a plant, a fungi, an animal). In particular embodiments, the eukaryote may be, but is not limited to, a microsporidia, a diplomonad, an oxymonad, a retortamonad, a parabasalid, a pelobiont, an entamoebae or a mitochondrial eukaryote (e.g., an animal, a plant, a fungi, a stramenopiles).

iv. Viruses

In certain embodiments the organism may be a virus. In particular aspects, the virus may be, but is not limited to, a DNA virus, including but not limited to a ssDNA virus or a dsDNA virus; a DNA RNA rev transcribing virus; a RNA virus, including but not limited to a dsRNA virus, including but not limited to a −ve stranded ssRNA or a +ve stranded ssRNA; or an unassigned virus.

c. Synthetic Samples

Nucleic acid samples comprising populations designed by the hand of man may also be generated and used as a standard against which another sample or subpopulation of target sequences could be compared. The synthetic population can be used to accurately quantify one or more targets from one or more sample(s) if the concentrations of the synthetic nucleic acids are known. For example, a synthetic sample may comprise a collection of nucleic acids (e.g., RNA, cDNA or genomic DNA) from many different tissues, cells (e.g., cell cultures), or other samples that could provide an average population against which a sample, or subpopulation of target sequences, could be compared. In another non-limiting example, the synthetic sample could comprise a collection of in vitro transcripts at known or unknown concentrations sharing a specific tag sequence so that they could be co-amplified with nucleic acids from another sample (e.g., RNA) to quantify a collection of targets. In another example, the synthetic sample could comprise a set of DNAs at known or unknown concentrations sharing a specific tag sequence that could be used to quantify a sample comprising a target DNA population.

d. Sample Mixtures

A sample mixture is a collection of two or more nucleic acid samples (e.g., RNA, cDNA or DNA). It is particularly preferred that the different nucleic acid samples (the “input samples”) that comprise the sample mixture are distinguishable. This is typically achieved by differentially tagging the targets of each input sample prior to mixing. In certain embodiments, a sample (e.g., an input sample) may comprise competitors. As used herein, a “competitor” is a nucleic acid (e.g., RNA or DNA) that can be amplified by the same primers used to amplify a target being assessed in a sample. In certain aspects, a competitor may be used to quantify a target by comparing the abundance of the amplified competitor to the abundance of the amplified target.

c. TAGS

In preferred embodiments, the invention involves appending a tag to one or more target sequences, up to all nucleic acid sequences, comprised in a nucleic acid population. A tag is a common sequence shared by nucleic acids within a sample that allows nucleic acids of one population to be distinguished from another sample. The term tag is also used to describe the RNA, DNA, or other nucleic acid molecule that is appended or otherwise used to attach a tag sequence to the nucleic acids comprising a sample. In preferred embodiments, a tag is an RNA, DNA, or other molecule that can be used as a template by a polymerase to generate a complementary strand.

A tag preferably comprises at least two functional domains, the first referred to as a “differentiation domain”, can be used to distinguish the nucleic acid targets derived from each sample (e.g., if there are multiple samples in a sample mixture). The second functional element, referred to as an “amplification domain,” is used to amplify nucleic acid target sequences. Thus, in preferred embodiments, a tag comprises at least two functional domains, an amplification domain compatible with amplification and a differentiation domain that can be used to distinguish nucleic acids that derive from the sample(s) being assessed. Of course, a tag may comprise one or more additional sequences.

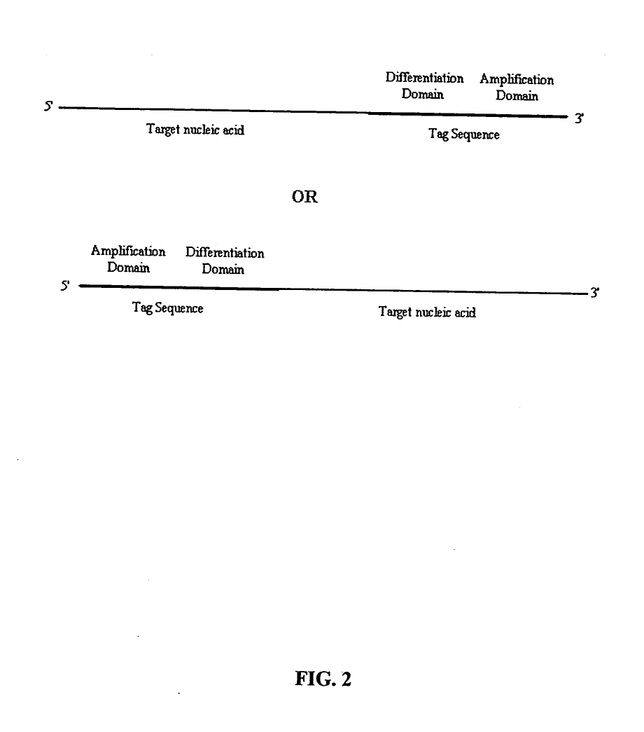

An example of a general makeup of a tag comprising an amplification and differentiation domain is provided in FIG. 2. As indicated in FIG. 2, it is particularly preferred that the differentiation domain is between an amplification domain and the sequence of each target nucleic acid of the population to be amplified. In other words, it is particularly preferred that a differentiation domain is internal to the amplification domain. Tag sequences can be appended either 5′ or 3′ of a nucleic acid target.

Although they are drawn as distinct domains (FIG. 2), the differentiation and amplification domain sequences can overlap, though it is particularly preferred that they are functionally distinct. It is also particularly preferred that the amplification domain sequence alone does not create interactions or reactions in subsequent steps that would foster amplified nucleic acids derived from one sample to be misinterpreted as derived from another sample.

1. Amplification Domains

In most embodiments, it is particularly preferred that a tag comprise at least one amplification domain. As used herein, an amplification domain will primarily be a sequence that can support the amplification of a nucleic acid that comprises such sequence. Use of nucleic acid sequences in amplification reactions are well known in the art, and non-limiting examples are described herein. The amplification domains of the tags used in samples that are mixed will preferably be identical to facilitate equal co-amplification of the target sequences from the different input samples that comprise the sample mixture being assessed.

In certain embodiments, an amplification domain will comprise a sequence that can support primer binding and extension. Standard rules for primer design apply (Sambrook, 1994). In specific aspects, an amplification domain will preferably comprise a primer binding sites for PCR™ amplification. PCR™ does not require any specialized structure or sequence to sustain amplification; the PCR™ amplification primer typically contains only binding sequences. Parameters for primer design for PCR™ are well known in the art (see, e.g., Beasley et al., 1999).

Primer binding sites for other types of amplification methods might also be used in amplification domains. Often such primer binding regions share similar characteristics with PCR™ primer binding sites, however the primers used for other amplification methods typically possess sequences 5′ to the binding domain. For instance, primers for 3SR and NASBA contain an. RNA polymerase promoter sequence 5′ to the priming site to support subsequent transcription. Because 3SR and NASBA are performed at relatively low temperature (37° C. to 42° C.), the amplification domains can have much lower melting temperatures than those used for PCR™.

2. Differentiation Domains

It is particularly preferred that a tag comprise at least one differentiation domain. A differentiation domain comprises a sequence that can be used to identify the sample from which a particular amplified nucleic acid derives. In a preferred aspect, each nucleic acid population being assessed will possess a differentiation domain comprising a unique sequence. Differentiation domains may comprise, but are not limited to, sequences such as hybridization/binding domains, unique sequences for sequence analysis or combinations thereof

a. Affinity Domains

In certain embodiments, a differentiation domain may provide an affinity site for hybridization or binding (an “affinity domain”) to a ligand comprising, but not limited to, a nucleic acid, protein or other molecule. An affinity domain may be used to distinguish from which sample a nucleic acid derives. In certain aspects, the affinity site may comprise a sequence that can hybridize to a nucleic acid ligand (e.g., an oligonucleotide or polynucleotide). In certain facets, the nucleic acid ligand may provide a method for identifying and quantifying nucleic acids possessing a sample-specific tag sequence. The sample specific ligands may be bound at unique addresses on an array, bound to different solid supports, or labeled with hybridization sensitive moieties, thus providing a method to measure the relative abundance of target sequences (e.g., amplified nucleic acids) comprising each different sample-specific tag sequence (U.S. Pat. Nos. 5,210,015 and 6,037,130).

b. Unique Sequence Domains

In embodiments where amplified nucleic acids from a target fraction are differentiated by sequence analysis, the differentiation domains should possess unique sequences. The abundance of amplified products derived from each sample can be determined by assessing the number of molecules possessing each unique sequence.

3. Additional Functional Domains

A tag may comprise one or more additional functional or structural sequences, as described herein or as would be known to one of ordinary skill in the art. In certain embodiments, these domains may be partly or fully comprised within other domains, such as, for example an amplification domain or a differentiation domain. In other embodiments, these additional domains may be comprised in sequences that do not comprise the amplification domain or differentiation domain.

These additional domain(s) may be used to support additional molecular biological reactions, including but not limited to an amplification reaction, a labeling reaction, a restriction digestion reaction, a cloning reaction, a hybridization reaction, sequencing reaction or a combination thereof.

Additional sequences described herein are by no means intended as an exhaustive list of all of the potential functional domains that can be included to facilitate production, amplification, differentiation, comparison or analysis of a nucleic acid target and/or sample. The list is merely intended to provide examples of some requirements and benefits of additional functional domains that can be incorporated into the nucleic acid tag.

a. Analysis Domains

In certain embodiments, a tag of the present invention further comprises a domain that provides a means for analyzing a nucleic acid population. Such analysis domains may comprise sequences that allow the production of detectable products from an amplified population or enable quantitative analysis of the amplified population. Such domains may comprise, but are not limited to, a labeling domain, a sequencing primer binding site or a restriction enzyme site to facilitate cloning and sequencing.

i. Labeling Domains

A tag may comprise a sequence that is used in a labeling reaction (a “labeling domain”) to convert an amplified nucleic acid population into a labeled product population for subsequent analysis. A variety of sequences can be used to support the production of labeled products, and non-limiting examples are described herein. In specific embodiments, a labeling domain may be used for labeled DNA or labeled RNA product synthesis. It is particularly preferred that the labeling domain be situated upstream of the differentiation domain so that the labeled population include the differentiation domain sequence.

Techniques for labeling the products of nucleic acid amplification are well known in the art (Sambrook, 1994) and include the incorporation of isotropically and non-isotopically labeled nucleotides during polymerization or the use of isotopically or nonisotopically labeled primers for primer extension.

In embodiments where it is desirable to generate labeled nucleic acid products following amplification, a primer binding site or transcription promoter sequence can be included in the tag between the amplification and differentiation domains. Of course, there may be sequence overlap between the labeling domain and the amplification domain, differentiation domain or both, as would be understood by one of skill in the art. The products of amplification may be used to initiate primer extension or transcription labeling reactions. For primer extension, an oligonucleotide complementary to the primer binding site can be incubated with the amplified population and extended by the action of a DNA polymerase. The primer can be non-isotopically or isotopically labeled or labeled dNTPs can be incorporated to generate labeled DNA.

In aspects wherein transciption is used to generate a labeled nucleic acid, the amplified products can be incubated with an appropriate polymerase and isotopically or nonisotopically labeled NTP(s) to create labeled RNA for analysis.

ii. Primer Binding Sites for Sequencing

A tag may comprise a primer binding site for sequencing in addition to any primer binding site in the amplification domain. For example, in certain preferred embodiments a primer binding site could be included in the tag sequence between the amplification and differentiation domains to facilitate sequence analysis of the differentiation domains of an amplified population.

iii. Restriction Enzyme Sites

A tag sequence may comprise one or more selected restriction enzyme sites, which may be used in various reactions, such as, for example, a cloning reaction. Methods of cloning are common in the art (Sambrook 1989). For example, cloning the amplified nucleic acid(s) resulting from competitive amplification will be particularly preferred to facilitate sequence analysis (e.g., quantitative sequence analysis). In a preferred embodiment, a target fraction from a sample mixture is amplified. The amplified nucleic acids are digested with restriction enzymes specific to the tag sequences shared by the targets. The digested DNA's may be cloned and sequenced. The differentiation domains whose sequence is unique for each sample are quantified to reveal the relative abundance of targets present in each of the input samples.

In certain preferred aspects, an amplified nucleic acid would comprise at least one restriction site on either side of a differentiation domain. In aspects wherein the restriction sites upstream and downstream of the differentiation domain were unique, then single differentiation domains could be directionally ligated into cloning vectors and subsequently sequenced.

In certain embodiments, restriction sites can be employed to facilitate concatenation for rapid sequence analysis as described in U.S. Pat. No. 5,866,330. For example, in aspects wherein the restriction sites were identical or otherwise able to be ligated, the differentiation domains could be ligated to one another to create extended chains of differentiation domains from amplified nucleic acids. In particular facets, the concatenized differentiation domains may be ligated into a cloning vector and subsequently sequenced to quantify the abundance of each differentiation domain in an amplified sample.

b. Secondary Amplification Domains

One or more amplification domains in addition to the primary amplification domain may be used for nested amplification (U.S. Pat. No. 5,340,728). In general embodiments, nested amplification comprises sequential amplification reactions wherein a first amplification with a first set of one or more primers generates one or more primary amplified nucleic acids, and at least a second amplification of the one or more primary amplified nucleic acids with another set of primers comprising a primer that binds a sequence partly or fully internal to a primer of the first set, so that a nucleic acid segment of the one or more primary amplified nucleic acids is then amplified. In certain embodiments, nested amplification might be required for those targets that are present in only a few copies in a sample or where small amounts of a sample (e.g., a few mammalian cells) are available. The secondary amplification domain is typically between the primary amplification domain and the differentiation domain of the tag.

D. Methods for Appending Tags to Populations

A nucleic acid tag of the present invention may be added to or appended to a nucleic acid population. As would be appreciated by one of ordinary skill in the art, different methods of tag attachment or incorporation may be used depending on whether the nucleic acid population comprises DNA or RNA. Non-limiting examples of such methods that may be used are described herein, though other methods can be used as would be known by one of ordinary skill in the art.

In addition to the techniques described herein, other methods of nucleic acid manipulation and/or additional compositions may be applied to nucleic acid targets, populations and/or samples. Such additional methods and compositions are described in detail in U.S. Patent Application No. 60/265,694, entitled “COMPARATIVE ANALYSIS OF NUCLEIC ACIDS USING POPULATION TAGGING,” filed on Jan. 31, 2001; U.S. Patent Application No. 60/265,695, entitled “COMPETITIVE POPULATION NORMALIZATION FOR COMPARATIVE ANALYSIS OF NUCLEIC ACID SAMPLES,” filed on Jan. 31, 2001; and U.S. Patent Application No. 60/265,693, entitled “METHODS FOR NUCLEIC ACID FINGERPRINT ANALYSIS” filed on Jan. 31, 2001, whose Ser. Nos. have not yet been assigned; each filed co-currently, and each of whose disclosure is specifically incorporated herein by reference in their entirety without disclaimer.

1. Tagging RNA

The methods of the present invention are applicable to tagging both eukaryotic RNA and/or prokaryotic RNA. In other aspects, the present invention may be applied to tag polyA selected or total RNA populations. As will be apparent to one of ordinary skill in the art in light of the disclosures herein, a tag may be appended to RNA populations in a variety of ways. Non-limiting examples of methods of tagging RNA are described below.

Once an RNA molecule is tagged, it can undergo further molecular biology reactions, including but not limited to, reverse transcription, hybridization and amplification. In preferred embodiments, the tagged RNA or cDNA population can be mixed with other tagged nucleic acids to create a sample mixture and targets within the sample mixture can be divided into target fractures. A target fraction may be obtained by binding nucleic acid targets to proteins or other molecules or by hybridizing nucleic acid targets to target specific sequences. The target fractions may then be amplified using tag and target specific primers. The amplified nucleic acids may be quantified using the unique differentiation domains to determine the abundance of target sequences in various samples.

a. Ligation

In certain embodiments, a tag can be appended to the 3′ ends of RNAs by a ligase (e.g., a protein, nucleic acid or chemical that induces ligation). For ligation, an excess of RNA or DNA polynucleotide tag possessing a 5′ phosphate can be added to a RNA population. Incubation of the mixture with a ligating agent (e.g., RNA ligase) generates RNAs with the tag ligated to the 3′ end of the RNAs.

In general embodiments, more efficient ligation may be achieved by adding bridging oligonucleotides to the ligation reaction. Hybridization of a bridge to both a nucleic acid to be tagged (e.g., an RNA or DNA in the sample) and a tag will align the 3′ and 5′ ends of the two molecules, enhancing ligation efficiency. In a non-limiting example, a bridging oligonucleotide may comprise a sequence at its 3′ end that is complementary to the 3′ end of the RNA population and a sequence at its 5′ end that is complementary to the 5′ end of the tag.

b. Cap Dependent Ligation

In one embodiment, a cap dependent ligation may be used to selectively append tags to the 5′ ends of eukaryotic mRNAs. In general aspects, an RNA may be tagged by the combined enzymatic activities of a phosphatase, a pyrophosphatase (e.g., a tobacco acid pyrophosphatase) that leaves a 5′ phosphate at the 5′ terminus of a capped message, and nucleic acid ligase (e.g., an RNA ligase).

In a non-limiting example, a total RNA population is treated with a phosphatase, such as calf intestinal phophatase (CIP), to dephosphorylate (i.e., remove the 5′ terminal phosphate cap structure of euakaryotic mRNA) of the RNA population. CIP is specific to RNAs with free terminal phosphates, therefore the 5′ phosphates of rRNAs, tRNAs, and partially degraded mRNAs are removed leaving these RNAs with 5′ hydroxyls. After the CIP is inactivated, the RNA preparation is treated with a phosphatase such as tobacco acid pyrophoshatase (TAP) to convert the 5′ cap structures of mRNAs to 5′ monophosphates. An excess of a DNA or RNA polynucleotide tag is added to the RNA population as well as a ligase that functions on RNA substrates. The tag should ligate exclusively to TAP modified RNAs possessing 5′ monophosphates as all of the non-capped RNAs possess 5′ hydroxyls following CIP treatment. The tagged mRNA sample can be used in subsequent reactions to fractionate, amplify and assess the samples' populations.

c. Enzymatic Polymerization

In an additional embodiment, a tag is incorporated into an RNA population by enzymatic polymerization. A tag with a 3′ nucleotide that cannot be extended by polymerization (see for example, U.S. Pat. No. 6,057,134), can be hybridized to the 3′ ends of an RNA population. An RNA or DNA polymerase with the ability to extend primer template junctions can be added to the mixture and allowed to extend the 3′ ends of the RNAs in the population, incorporating a sequence complementary to the hybridized oligonucleotide at the 3′ ends of the RNA in the sample. If the nucleic acid that serves as a template comprises a tag sequence, then the polymerization reaction effectively tags the nucleic acid sample population.

d. CAPswitch™

A method for tagging mRNAs by Cap-induced primer extension is described in U.S. Pat. No. 5,962,271. The technology, referred to as CAPswitch™, uses a unique CAPswitch oligonucleotide in the first strand cDNA synthesis reaction. When reverse transcriptase stops at the 5′ end of an mRNA template in the course of first strand cDNA synthesis, it switches to a CAPswitch oligonucleotide and continues DNA synthesis to the end of a CAPswitch oligonucleotide. The resulting cDNA has at its 3′ end a sequence that is complementary to the CAPswitch oligonucleotide sequence. The CAPswitch technology may be used to tag one or more RNA populations by using one or more CAPswitch oligonucleotides comprising differentiation and amplification domains.

2. Tagging RNA Populations by Reverse Transcription

In a preferred embodiment, tag sequences may be appended to sample nucleic acids by reverse transcription. For example, tagged cDNA populations can be conveniently generated by priming reverse transcription with oligonucleotides tags comprising an amplification and an differentiation domain at its 5′ end and a target-specific priming domain at its 3′ end. Hybridization of the primer to one or more species in an RNA sample and subsequent reverse transcription yields cDNA with tag sequences at its 5′end.

For example, most eukaryotic mRNAs possess a polyA tail that can hybridize to a primer that has polyT or polyU at or near its 3′ end and amplification and differentiation domains at its 5′end. The polyA specific tag primer can be extended from the polyA tail of the mRNAs. The resulting cDNAs would possess the appended tag sequences at or near their 5′ ends that may be used in one or more amplification or differentiation reactions. In certain aspects, cDNA populations that can be mixed, divided into target fractions, and amplified for quantitative analysis.

3. Tagging Prokaryotic RNA Samples

The methods described above may be less preferred for tagging a non-polyA RNA, such as a prokaryotic RNA. However, analysis of prokaryotic RNA samples is desirable in certain aspects.

In a non-limiting example, it may be desirable to remove or separate small RNAs (e.g., tRNAs) from prokaryotic mRNA or an RNA population lacking a polyA tail. Methods of removing small RNA are known to those of skill in the art, and include such methods as a lithium chloride precipitation. Lithium chloride precipitation is specific to RNAs greater than 100-300 nucleotides, thus tRNAs and other small RNAs will be removed from the RNA population (Sambrook 1989).

In embodiments wherein non-rRNAs are the target RNAs, one or more rRNA specific oligonucleotides and a polyA polymerase can be used to add polyA tails to prokaryotic mRNAs. For example, a sample comprising prokaryotic total RNA can be precipitated with lithium chloride. After removal of the solution comprising the tRNAs, a resulting RNA population can be resuspended and hybridized to one or more oligonucleotides complementary to the 3′ ends of the major prokaryotic rRNAs. The 5′ ends of the oligonucleotide(s) will preferably extend beyond the 3′ ends of the rRNAs, creating a slight 5′ overhang. The RNA population can then be treated with a polyA polymerase and ATP. RNAs with non-hybridized 3′ ends can be extended by the action of the polymerase, creating a 3′ polyA tail on the mRNA portion of the sample. The resulting polyA modified RNA can then be reverse transcribed using a tag comprising a oligo-dT and amplification and fingerprint domains.

4. Tagging DNA

DNA (e.g., genomic DNA and cDNA) can be tagged by various methods, including primer extension or ligation. In certain aspects, the DNA may be single stranded or double stranded.

a. Single Stranded DNA

In one embodiment, a target single-stranded DNA (e.g., cDNA) population may be diluted in a buffer appropriate for hybridization and polymerization, and hybridized to one or more tags. Addition of a DNA polymerase such as, for example, the klenow fragment of DNA polymerase I or Taq DNA polymerase, will extend the hybridized tag to create a tagged population of DNA segments.

In aspects where the DNA is double stranded (e.g., genomic DNA), it may be denatured prior to tagging by any of a variety of methods known in the art, including, for example, heating to 95° C. in a solution of 0.2 M NaOH. In certain aspects, the denatured DNA may be removed or purified from the denaturing reagents by methods well known to those of skill in the art, such as, for example, ethanol precipitation. The denatured DNA may then be tagged using techniques described herein or as would be known to one of ordinary skill in the art.

b. Double Stranded DNA

In certain embodiments, double stranded DNA may be tagged by ligation. For example, a double-stranded DNA can be digested with a restriction enzyme, and one or more double stranded tags comprising a compatible restriction fragment cut site may be ligated to the digested DNA. It is particularly preferred that the unligated tags be removed prior to amplification to keep them from participating in the amplification reaction.

A disadvantage of appending double-stranded tags to double-stranded nucleic acids (e.g., DNA) is that primers specific to the amplification domain of the tag can bind and be extended from target and non-target molecules alike. Using restriction digestion and double-stranded tag ligation may create far greater background than the other methods and is therefore a less preferred method for tagging populations. This is in contrast to other tagging methods described herein, whereby single-stranded tags are appended to single-stranded nucleic acids from the sample. In these embodiments, the amplification domain of the tag sequence only becomes a primer binding site when the target specific primer is extended during the amplification phase.

E. Separating Nucleic Acids into Sample Fractions

A particularly preferred step in the methods of the present invention comprises the sequence-dependent fractionation of nucleic acids in a sample, particularly sample mixtures, prior to quantitative analysis (e.g., amplification and/or differentiation reactions). Dividing the sample into sample fractions increases the number of targets that can be characterized from a sample and increases the specificity of the competitive amplification reaction. The targets that comprise a target fraction can be assessed using the methods described herein or as would be known to one of skill in the art.

Removing a target fraction from a sample mixture can be achieved by a variety of methods, described herein or as would be known to those of ordinary skill in the art in light of the present disclosures. For example, in certain embodiments, fractionating target nucleic acids is conveniently accomplished by binding a target specific ligand. In general aspects, the ligand is bound to an array or other solid support. In a preferred embodiment, fractionation may be achieved by hybridizing complementary nucleic acids to nucleic acid targets within a sample mixture.

In further aspects, the hybridized or bound nucleic acids may be extracted from the array or other solid support and subjected to further analysis reactions, such as amplification and differentiation reactions. An amplified population could then be used for subsequent quantitative analysis to determine what percentage of amplified nucleic acids derived from each of the samples, comprise the sample mixture.

1. Arrays

Gene arrays are solid supports upon which a collection of target-specific ligands (e.g., probes) has been attached (e.g., spotted) at defined locations. For example, the probes may localize complementary nucleic acid (e.g., RNA or DNA) targets from a nucleic acid sample by hybridization. Because the number of ligands that can be spotted on a gene array is virtually unlimited, arrays can be used to fractionate tens of thousands of target nucleic acid molecules from one or more nucleic acid samples.

The amount of a target that becomes bound at each spot is a function of the amount of the target present in the sample population. Thus, in embodiments wherein different samples are being hybridized to a single array, the amount of target nucleic acid from one sample hybridizing at each ligand's spot is a function of the relative abundance of that target in the sample compared to the other sample(s).

In certain embodiments, nucleic acid (e.g., RNA or DNA) samples would be mixed and the resulting sample mixture incubated with one or more gene arrays. In certain aspects, individual spots on the array may be excised to provide a target fraction.

2. Other Solid Supports

Several methods have been developed that, like array analysis, differentiate targets by sequence-specific hybridization. It is contemplated that these methods, described herein, and others known to those of skill in the art, may be applied in the methods of the present invention, in light of the present disclosures.

a. Beads

In some embodiments, (U.S. Pat. No. 5,981,180) target-specific oligonucleotides can be appended to fluorescent or otherwise distinguishable beads. A sample mixture can be incubated with the beads to allow target nucleic acids to hybridize to appropriate bead-bound oligonucleotides. The beads, which are unique for each target, can be fractionated to generate target factions comprising populations of specific targets. The nucleic acid targets in each fraction can then be amplified using tag and target specific primers and the amplification products assessed.

b. Chip Based Fractionation

Although it is preferred that multiple targets be fractionated simultaneously, a sample mixture can also be analyzed for one target at a time. A ligand specific to a single target can be incubated with the nucleic acid sample. After the target fraction hybridizes to the nucleic acid, the nucleic acid can be removed from the sample mixture. This may be accomplished by appending the ligand (e.g., a nucleic acid) to a bead or other solid support prior to incubation with the sample mixture. Alternatively, the ligand could be biotinylated or otherwise modified within an affinity site. The ligand may be removed from the sample with a molecule that binds the affinity site (e.g., streptavidin or otherwise derivatized solid support) following target hybridization. Additional modifications known to those of skill in the art could be used to allow hybridized targets to be removed from the sample population.

In another non-limiting example, chip-based formats may also be used to fractionate target nucleic acids by hybridization to oligonucleotides at discrete locations (U.S. Pat. Nos. 5,632,957 and 5,955,028, each incorporated herein by reference). These methods typically incorporate microchannels etched in silicon wafers. Samples comprising nucleic acid populations pass through the channels under the guidance of electrical fields. Interactions between target nucleic acids and complementary oligonucleotides along these channels localize one or more targets to discrete locations on the chips.

F. AMPLIFICATION

In certain preferred embodiments of the invention, nucleic acid amplification is employed to generate detectable amounts of target-specific nucleic from target fractions. A variety of methods have been described for nucleic acid amplification, and are known to those of skill in the art

Although slight variations abound, the general principle of amplifying nucleic acids is the same. In embodiments for amplifying DNA, a sample comprising a DNA population is contacted with one or more amplification primers that are able to hybridize to targets comprising the DNA population with amplification reagents in appropriate amplification conditions. The amplification primers may comprise, for example, a primer specific to the amplification domain of the sample's population, and a primer specific to a sequence within the target nucleic acids.

1. Polymerase Chain Reaction

A number of template dependent processes are available to amplify sequences present in a given sample. A non-limiting example is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety. Other non-limiting methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety. PCR could be used to amplify sample mixtures comprising tagged DNA and cDNA samples using one primer specific to the amplification domain of the tag and one primer specific to the target sequence.

In another embodiment, a reverse transcriptase PCR™ amplification procedure may be performed to amplify mRNA populations. Methods of reverse transcribing RNA into cDNA are well known (see Sambrook, 1989). Alternative methods for reverse transcription utilize thermostable DNA polymerases. These methods are described in WO 90/07641. Additionally, representative methods of RT-PCR are described in U.S. Pat. No. 5,882,864. Reverse transcriptase PCR could be used to amplify a sample mixture comprising tagged RNA samples using one primer specific to the amplification domain of the tag and one primer specific to the target sequence.

2. Nucleic Acid Sequence Based Amplification

Other non-limiting nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

Nucleic Acid Sequence Based Amplification (NASBA) (Guatelli, 1990; Compton, 1991) makes use of three enzymes, avian myeloblastosis virus reverse transcriptase (AMV-RT), E. coli RNase H, and T7 RNA polymerase to induce repeated cycles of reverse transcription and RNA transcription. The NASBA reaction begins with the priming of first strand cDNA synthesis with a gene specific oligonucleotide (primer 1) comprising a T7 RNA polyerase promoter. RNase H digests the RNA in the resulting DNA:RNA duplex providing access of an upstream target specific primer(s) (primer 2) to the cDNA copy of the specific RNA target(s). AMV-RT extends the second primer, yielding a double stranded cDNA segment (ds DNA) with a T7 polymerase promoter at one end. This cDNA serves as a template for T7 RNA polymerase that will synthesize many copies of RNA in the first phase of the cyclical NASBA reaction. The RNA copies then serve as templates for a second round of reverse transcription with the second gene specific primer, ultimately producing more DNA templates that support additional transcription.

In certain embodiments, NASBA could be adapted to the present invention to provide competitive amplification of target sequences. For example, the amplification domain of the tag sequence would comprise a promoter for an RNA polymerase and a primer binding site downstream of the promoter. A nucleic acid primer would initiate amplification by driving complementary strand synthesis from a target sequence. If the sample mixture comprised DNA, then the resulting double-stranded nucleic acid would be a template for transcription. If the sample mixture comprised RNA, then a primer specific to the amplification domains of the samples would bind the cDNA of the first strand reaction and prime synthesis of a double-stranded template. In either case, the double stranded DNA would be trancribed by the action of the RNA polymerase and the resulting transcripts would be reverse transcribed and further converted to transcription templates by the actions of the primers and enzymes in the NASBA reaction. The amplified nucleic acids (e.g., RNA or cDNA) could be quantified using the unique differentiation domains of the appended tags. The ratio of amplified nucleic acids with each different differentiation domain would reflect the relative abundance of the target sequence in the samples.

3. Strand Displacement Amplification

Strand Displacement Amplification (SDA) is an isothermal amplification scheme that consists of five steps: binding of amplification primers to a target sequence, extension of the primers by an exonuclease deficient polymerase incorporating an alpha-thio deoxynucleoside triphosphate, nicking of the hemiphosphorothioate double stranded nucleic acid at a restriction site, dissociation of the restriction enzyme from the nick site, and extension from the 3′ end of the nick by an exonuclease deficient polymerase with displacement of the downstream non-template strand. Nicking, polymerization and displacement occur concurrently and continuously at a constant temperature because extension from the nick regenerates another hemiphosphorothioate restriction site. In embodiments wherein primers to both strands of a double stranded target sequence are used, amplification is exponential, as the sense and antisense strands serve as templates for the opposite primer in subsequent rounds of amplification.

In some embodiments, SDA may be adapted to the present invention to provide competitive amplification of target sequences. For example, the amplification domain of the tag sequence would comprise a primer binding site and an appropriate restriction enzyme site. A sample mixture may be added to an SDA reaction with tag and target specific primers with associated restriction sites compatible with SDA. The primers could be extended and the extended nucleic acids could be digested by restriction enzymes specific to the restriction sites in the tag and target primers. The digested nucleic acids would serve as templates for subsequent cycles of primer extension and restriction digestion. The final amplified nucleic acids would be assessed to determine the relative abundance of amplified nucleic acids possessing each of the sample-specific differentiation domains.

4. Transcription

DNA molecules with a double-stranded transcription promoter can be templates for any one of a number of RNA polymerases (Sambrook 1989). An efficient in vitro transcription reaction can convert a single DNA template into hundreds and even thousands of RNA transcripts. While this level of amplification is orders of magnitude less than what is achieved by PCR™, NASBA, and SDA, it could be sufficient for some embodiments of the present invention.

In certain embodiments, to use transcription as an amplification step in the present invention, the amplification domains of the tags appended to the samples being assayed comprise identical transcription promoters. For example, the single-stranded cDNA from targets could be converted to double-stranded transcription templates using a primer comprising a target specific domain, a polymerase, and deoxynucleoside triphosphates. The resulting templates could be added to an in vitro transcription reaction with a polymerase appropriate to the promoter sequence of the tag amplification domain. Following transcription, the differentiation domains of the RNA population may be quantified to determine the relative abundance of the target in each of the nucleic acid samples.

5. Rolling Circle Amplification

Rolling circle amplification has been used to detect target nucleic acids (Lizardi, 1998; Zhang, 1998). This amplification reaction uses a circular nucleic acid template. Linear templates are typically circularized by hybridizing the 5′ and 3′ ends of the template to a single nucleic acid molecule that brings the terminal template nucleotides into close proximity. A ligase is added to circularize the template. A primer complementary to the circular RNA or DNA then hybridizes and initiates primer extension. Using a polymerase with strand-displacing activity allows the extended nucleic acid to be infinitely long. To achieve exponential amplification, a primer specific to the displaced ssDNA nucleic acid is added to the reaction. Multiple copies of the second primer can hybridize along the length of the Rolling Circle product nucleic acid. Extension and strand displacement at the multiple sites produces complementary molecules. Priming off of these nucleic acids by the first primer contributes to the accumulation of target dependent nucleic acid synthesis.

In some embodiments, Rolling Circle Amplification may be adapted to the present invention to provide competitive amplification of the target fractions. For example, for each target being assayed, a polynucleotide would be synthesized that possessed sequence at its 3′ end that is complementary to the 5′ end of the tag sequence and at its 5′ end sequence complementary to the 3′ end of the target cDNA. Following hybridization to the targets in the sample mixture, the target cDNA would be ligated to circularize the template. A primer specific to the amplification domain of the appended tags would be added to initiate rolling circle amplification. The amplified nucleic acids may be quantified to determine the relative number of differentiation domains derived from each input sample in order to determine the abundance of the target in each of the input samples.

G. Differentiation Reactions

A differentiation reaction may comprise any of a number of methods that distinguish from which sample a particular amplified nucleic acid derives. In certain embodiments, the proportion of amplified nucleic acids derived from each of the samples can be determined.

1. Affinity Sites

A differentiation domain of a tag may comprise a sequence with an affinity for a specific nucleic acid, protein, or other binding ligand. A binding ligand may comprise, but is not limited to, an oligonucleotide complementary to a differentiation domain, a nucleic acid binding protein (e.g., a transcription factor) that binds to a specific DNA or RNA sequence, a small molecule that intercalates into a given RNA or DNA sequence or combinations thereof. A binding ligand may either be bound to a solid support (e.g., a single bead or an array) or otherwise readily removed or separated from a solution.

In embodiments wherein hybridization or binding to support-bound ligands (e.g., nucleic acids) is being used for quantification, the amplified nucleic acids should be labeled directly or used to generate a labeled population. As described herein, labeling can be accomplished during the amplification reaction by incorporating labeled nucleotides or primers. Alternatively, sequences within the amplified nucleic acids, especially in the tag sequence, can be used to generate labeled RNA or DNA for ligand hybridization and quantification.

In specific aspects, labeled nucleic acids would be applied to a solution or solid support possessing ligands specific to the differentiation domains of the different samples.

In certain preferred embodiments, differentiating (e.g. separating and quantifying) amplified nucleic acids may be accomplished using an array comprising nucleic acids that would be complementary to each of the differentiation domains appended to the samples being assessed. The labeled nucleic acids generated by amplification of a given target fraction could be incubated with the array under conditions that promote hybridization. Each distinct differentiation domain could hybridize to the appropriate address on the array. The hybridized amplification products could be quantified to determine the percentage of amplified targets derived from each sample population. Because every target within the sample mixture possesses the same collection of sample-specific differentiation domains, the makeup of the array would be identical for each target being characterized.

A variety of techniques related to array analysis employ support-bound nucleic acids to distinguish and quantify target nucleic acids possessing unique sequences. Technologies described in U.S. Pat. Nos. 5,981,180, 5,632,957 and 5,955,028 could all be used to characterize the amplified nucleic acids generated while practicing the present invention. One skilled in the art will understand that many other similar technologies have also been described and would likewise facilitate the analysis of nucleic acids in the methods of the present invention.

2. Differentiation by Sequence Analysis

Because the sequences of the differentiation domains are unique, methods for sequence analysis that are known in the art could be used to assess the population of amplified nucleic acids to determine the relative abundance of targets present in each sample. In embodiments wherein a few samples are being assayed, the population of amplified nucleic acids could be easily sequenced. The relative abundance of each differentiation domain could be determined by measuring the relative intensity of bands at each sequencing position. Provided that the positions being quantified were unique for each differentiation domain, the band intensity for each different nucleotide in the peak would correspond to the relative abundance of that amplified target nucleic acid in the sample.

A simpler, though more laborious method for quantifying amplified nucleic acids by sequence analysis involves cloning and sequencing. The amplified nucleic acids would be ligated into cloning vectors, the resulting plasmids would be used to transform a suitable host such as E. coli, the transformed sample would be used to isolate clones, and the clones would be sequenced using methods common in the art (Sambrook 1989). The number of clones possessing each differentiation domain would be tallied to reveal the make-up of the amplified population.

Various methods may be used for cloning amplified nucleic acids. For example, if one or more restriction sites are present on either side of the differentiation domain, then the amplified nucleic acids and an appropriate cloning vector could be digested with appropriate restriction enzymes and ligated together prior to transformation.

In another embodiment, cloning of amplified nucleic acids may be accomplished without the use of restriction digestion. For example, U.S. Pat. No. 5,487,993 takes advantage of the activity of many thermostable polymerases whereby a non-templated dATP is attached to the 3′ ends of PCR™ amplified nucleic acids. The PCR™ amplified nucleic acids can be readily ligated into linearized vectors possessing single T overhangs at their 3′ ends without restriction digestion of the amplified nucleic acids. It is contemplated that this method could be incorporated into the present invention by providing a rapid method to clone the amplified nucleic acids for each target sequence. The cloned amplified nucleic acids could be sequenced using any of the methods common in the art.

U.S. Pat. No. 5,695,937 describes another technique that could facilitate the sequencing of amplified nucleic acids generated in the practice of the present invention. Serial analysis of gene expression (SAGE) is a method that allows for the rapid quantitative analysis of independent nucleic acids. The method involves digesting DNA populations with restriction enzymes that generate short, double-stranded oligomers. The oligomers are ligated together, cloned, and sequenced. A single sequencing run can provide the identity of 20 to 50 oligomers, for example. Because each oligomer represents a unique member of the sample's DNA population, the makeup of a nucleic acid sample can be determined. Several sequencing runs can provide statistically significant quantitative data on the relative abundance of the targets that comprise a nucleic acid sample.

SAGE could facilitate the quantitative analysis of amplified populations generated by protocols incorporating the methods of the present invention. To use SAGE, tag sequences would preferably comprise appropriate restriction sites upstream and/or downstream of the differentiation domains. The amplified population would be digested with restriction enzymes, the differentiation domains would be concatenized and cloned, and the clones would be sequenced. The sequenced differentiation domains would be quantified to reveal the relative abundance of target sequences in each of the samples.

3. Differentiation by Hybridization in Solution

In other embodiments, differentiating the amplification products may be conducted in solution. For example, U.S. Pat. Nos. 5,210,015 and 6,037,130 describe techniques that detect an amplified nucleic acid possessing specific sequences. Either of these two methods could be used to quantify amplified targets generated with the methods of the present invention. In one embodiment, oligonucleotides (e.g., labeled probes) specific to each of the differentiation domains present in the tags of a particular collection of samples could be synthesized and hybridized to an amplified population. The amount of signal from each different oligonucleotide would reveal the relative abundance of each sample-specific differentiation domain. In embodiments wherein the differentiation domain-specific oligonucleotides are labeled with the same or indistinguishable detectable moiety, it is particularly preferred that the differentiation domains be assessed separately with each of the different oligonucleotides. Alternatively, oligonucleotides labeled with distinguishable moieties could be used in a single detection reaction to quantify multiple differentiation domains in a single sample. The latter method would be preferred as it facilitates rapid sample analysis.

Other methods of nucleic acid detection that may be used in the practice of the instant invention are disclosed in U.S. Pat. No. 5,840,873, which describes detection of multiple nucleic acids utilizing oligonucleotide probes coupled to different chemiluminescent labeling reagents; U.S. Pat. No. 5,843,651, which describes an method for detecting nucleic acid sequence differences through binding to a ligand; U.S. Pat. No. 5,846,708 describes an array based binding and detection system; U.S. Pat. No. 5,846,717, describes detection systems based on nucleic acid cleavage; U.S. Pat. No. 5,846,726, describes nucleic acid probes that detects nucleic acids by changes in fluorescence of the a probe attached dye after cleavage of a nucleic acid; U.S. Pat. No. 5,846,729 describes nucleic acid detection in solution using changes in fluorescence of probes; each of which is incorporated herein by reference.

H. Identifying a Tag

Because unique tags are used for different sample populations, it will be very important that the unique tags not contribute to amplification or differentiation biases (e.g. differences in amplification or differentiation efficiencies). In certain embodiments, it is preferred that the differentiation domains provide equal levels of hybridization for amplification products from each sample.

If two or more different tags are used to compare nucleic acid samples, the new tag sequences can be compared to ensure that they function equivalently. The most powerful experiment contemplated for such a comparative test involves splitting a single sample into multiple tagging reactions incorporating the different tags. After tagging, the differentially tagged samples can be mixed, fractionated, amplified, and differentiated.

The differentiated nucleic acids are assessed by using the method that is to be applied for analysis. For example, if array analysis is to be used to distinguish differentiated nucleic acids derived from each sample, then the labeled nucleic acids resulting from amplification may be hybridized to the array. The signal from equivalently functioning tags at the addresses of the array should be equal because the amount of target for each sample is identical.

### I. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Arrays to Fractionate Targets for Competitive Amplification

A collection of RNA standards (Armored RNA constructs IL-1b, IL-2, IL-3, IL-4, IL-5, and IL-7 from Ambion) was heated to 75° C. for ten minutes. The RNAs were cooled to room temperature and reverse transcribed in 10 μl reactions containing 5 μM random sequence decamers, reaction buffer (50 mM Tris pH 8.3, 75 mM KCl, 3 mM MgCl₂, 5 mM DTT), and 20 U/μl MMLV-RT. The cDNA samples were placed in separate PCR™ reactions containing 500 μM dNTPs, reaction buffer (75 mM KCl, 50 mM Tris pH 9.0, 2 mM MgCl₂), and 2.5 units SuperTaq polymerase. 200 nM of the target specific primer pairs shown in Table 2 was added to the appropriate reactions and 20 cycles of PCR™ amplified the target inserts.

TABLE 2
PCR™ primers used to amplify probes for array analysis

IL-1 beta Primers

| | | |
|---|---|---|
| SEQ ID NO:1 | 5′ primer: | GGTGTTCTCCATGTCCTTTGT |
| SEQ ID NO:2 | 3′ primer: | TTGGGGAACTGGGCAGACTCA |
| IL-2 Primers | | |
| SEQ ID NO:3 | 5′ primer: | CAAACCTCTGGAGGAAGTGCTAA |
| SEQ ID NO:4 | 3′ primer: | GTGGGAAGCACTTAATTATCAAG |
| IL-3 Primers | | |
| SEQ ID NO:5 | 5′ primer: | CCTTGTGCGGTTGTGTTCTCATT |
| SEQ ID NO:6 | 3′ primer: | TCTCACACATCCCTAGGAACCAG |
| IL-4 Primers | | |
| SEQ ID NO:7 | 5′ primer: | TGCTGCCTCCAAGAACACAACTG |
| SEQ ID NO:8 | 3′ primer: | CATGATCGTCTTTAGCCTTTCCA |
| 11-5 Primers | | |
| SEQ ID NO:9 | 5′ primer: | TCGAACTCTGCTGATAGCCAA |
| SEQ ID NO:10 | 3′ primer: | GCAGTAAAATGTCCTTCTCCTCC |
| IL-7 Primers | | |
| SEQ ID NO:11 | 5′ primer: | GTGAAGCCCAACCAACAAAGAG |
| SEQ ID NO:12 | 3′ primer: | TTGGAGGATGCAGCTAAAGTTC |

The PCR™ products were denatured in 0.2 M NaOH/10 mM EDTA by heating to 95° C. for ten minutes. The denatured PCR™ products were spotted at defined locations on positively charged nylon membranes. The membranes were dried and cross-linked using a StrataLinker (Stratagene) set to 120 mJoules.

### Example 2

### Preparation of Arrays for Differentiating Sample Specific Amplification Products

A T7 oligonucleotide CTGTAATACGACTCACTATAGGGAGA (SEQ ID NO:13) and a SP6 oligonucleotide CTGATTTAGGTGACACTATAGAAGAGT (SEQ ID NO:14) were spotted at defined locations on positively charged nylon membranes. The membranes were dried and cross-linked using a StrataLinker (Stratagene) set to 120 mJoules.

### Example 3

### Tag Sequences Appended Via Reverse Transcription

This example describes one embodiment wherein a tag comprising a differentiation domain that includes a promoter (e.g., either a T7 or SP6 promoter) was appended to cDNA using reverse transcription.

1 μg of thymus total RNA or liver total RNA was mixed with 0.5 mM dNTPs, 50 nM T7-VN-1 or SP6-VN-1, and reaction buffer (50 mM Tris pH 8.3, 75 mM KCl, 3 mM MgCl₂, 5 mM DTT). The T7-VN-1 and SP6-VN-1 reverse transcription primers were are as follows:

| | |
|---|---|
| T7-VN-1 | |
| GCTGATGGCGATGAATGAACACTGTAATACGACTCACTATAGGGAGATTTTTTTTTTT | (SEQ ID NO:15) |
| TVN. | |
| SP6-VN-1 | |
| GCTGATGGCGATGAATGAACACTGATTTAGGTGACACTATAGAAGAGTTTTTTTTTTT | (SEQ ID NO:16) |
| TVN. | |

In regard to these reverse transcription primer sequences:

Bold=Amplification Domain,

Italics=Differentiation Domain, and

Plain Text=Anchored oligodT.

The mixture was heated to 68° C. for five minutes, then cooled to 42° C. RNase inhibitor (40 units/μl) and Moloney-Murine Leukemia Virus-Reverse Transcriptase (M-MLV-RT) (20 units/μl) were added and the mixture was incubated at 42° C. for one hour to convert the RNA samples to tagged cDNAs.

To remove the reverse transcription primers, the samples were applied to S-200 HR sephacryl spin columns. The columns were spun at 700×G for 2 minutes. The filtrate was recovered, providing the tagged cDNA population used for subsequent analyses.

### Example 4

### Tag Sequences Appended Via Cap-Dependent Ligation

This example describes how an RNA can be tagged via cap-dependent ligation. Of course, those of skill in the art will understand that there are additional manners of appending tag sequences, as disclosed in this specification and/or known in the art.

1 μg of mouse thymus or liver total RNA was treated with 1 unit of Calf Intestine Phosphatase in a 20 μl reaction of 50 mM Tris pH 8.5 and 0.1 mM EDTA to remove 5′terminal phosphates from uncapped RNAs. The-reaction was incubated at 37° C. for 1 hr. The volume of the reaction was increased to 150 μl by the addition of 500 mM Ammonium acetate, 1 mM EDTA. One acid phenol/chloroform and one chloroform extraction were done. The RNA was then precipitated and air dried.

To convert the capped mRNA in the sample to RNA with 5′monophosphates, the dephosphorylated RNA population was dissolved in 8 μl of water. 1 μl of 10× TAP buffer (500 mM NaOAc, 10 mM EDTA, 1% beta-mercaptoethanol, 0.1% CHAPS) and 0.1 unit of TAP were added and the decapping reaction was allowed to proceed for 1 hour at 37° C. Ammonium acetate was added to the reaction to provide a final concentration of 0.5 M. A two-fold volume of ethanol was added and the RNA was precipitated by incubation at −20° C. for fifteen minutes. The precipitated RNA was recovered by centrifugation.

To ligate the primers to the RNA possessing a 5′ monophosphate, the RNA pellet was dissolved in 8 μl of water. 1 μl of 10× ligation buffer (500 mM Tris pH 7.8, 100 mM MgCl₂, 100 mM DTT, 1 mM ATP), 1 μg of the ligation tag, and 5 units of T4 RNA ligase were added. The ligation tag sequence is shown, with the amplification domain in bold and the differentiation domain in italics: 5′TAATACGACTCACTATAGGGTTCGGGCTTAGGCTCCAGTGCCTGTTCGGTGGTCGC GGCGCTGATGGCGATGAATGAACACTGCGGCAAGCCGCTTAATGACACTCGTTTGC TGGCTTGATGGGCGAGCTGGAAGGCCGTATCTCCGGCAGCATTCATTACGACAAA3′ (SEQ ID NO:17). The ligation reaction was allowed to proceed for 1 hour at 37° C.

To remove the unincorporated ligation tags, the samples were applied to S-200 HR sephacryl spin columns. The columns were spun at 700×G for 2 minutes. The filtrate was recovered, providing the tagged cDNA population used for subsequent analysis.

### Example 5

### Fractionating Target Sequences for Differentially Tagged cDNA Samples

The differentially tagged cDNA samples from Example 3 were mixed and prepared for array analysis by heat denaturing in 100 μl of 10 mM EDTA. The cDNA was added to the array described in Example 1 that had been prehybridized for 30 minutes at 42° C. in hybridization buffer (2.5×SSC, 50% formamide, 7% SDS, 200 μg/ml yeast RNA). The array and cDNA were incubated overnight at 42° C. in hybridization buffer, then washed 2×30″ at 42° C. in 2×SSC/0.5% SDS and 2×30″ at 60° C. in 0.5×SSC/0.5% SDS. The spots corresponding to different target-specific polynucleotides were removed to separate tubes for subsequent analysis.

### Example 6

### Fractionating Target Sequences from Differentially Tagged mRNA Samples

The RNA from Example 4 was added to the array described in Example 1 that had been prehybridized for 30 minutes at 42° C. in hybridization buffer (2.5×SSC, 50% formamide, 7% SDS, 200 μg/ml yeast RNA). The array and RNA were incubated overnight at 42° C. in hybridization buffer, then washed 2×30″ at 42° C. in 2×SSC/0.5% SDS and 2×30″ at 60° C. in 0.5×SSC/0.5% SDS. The spots corresponding to different target-specific polynucleotides were removed to separate tubes for subsequent analysis.

### Example 7

### Competitive-PCR of Fractionated cDNA Targets

The cDNA fractions from Example 5 were placed in tubes with 10 μl of 10 mM EDTA and heated to 95° C. for ten minutes. 2 μl of each sample was added to the appropriate amplification reaction. Each PCR™ reaction comprised 100 nM of the tag primer and one of the target-specific primers (Table 3), 100 μM dGTP, dTTP, dCTP, 0.5 μM dATP and 0.5 μM [α-³²P]-dATP, reaction buffer (75 mM KCl, 50 mM Tris pH 9.0, 2 mM MgCl₂), and 2.5 units SuperTaq polymerase.

TABLE 3
Tag and Target Specific Primers

| | |
|---|---|
| Tag primer (SEQ ID NO:18): | GCTGATGGCGATGAATGAACACTG |
| Target Specific Primer 1 | GGTGTTCTCCATGTCCTTTGT |
| (SEQ ID NO:19): | |
| Target Specific Primer 2 | CAAACCTCTGGAGGAAGTGCTAA |
| (SEQ ID NO:20): | |
| Target Specific Primer 3 | CCTTGTGCGGTTGTGTTCTCATT |
| (SEQ ID NO:21): | |
| Target Specific Primer 4 | TGCTGCCTCCAAGAACACAACTG |
| (SEQ ID NO:22): | |
| Target Specific Primer 5 | TCGAACTCTGCTGATAGCCAA |
| (SEQ ID NO:23): | |
| Target Specific Primer 6 | GTGAAGCCCAACCAACAAAGAG |
| (SEQ ID NO:24): | |

PCR™ amplification was conducted by thirty cycles the following profile: 94° C., 30 seconds, 55° C., 30 seconds, 72° C., 60 seconds. At the end of the final amplification cycle, a 5 minute, 72° C. soak was used for annealing and primer extension.

### Example 8

### Competitive RT-PCR of Fractionated mRNA Targets

The RNA fractions from Example 6 were placed in tubes with 10 μl of 10 mM EDTA and heated to 95° C. for ten minutes. 2 μl of each sample was added to separate tubes and reverse transcribed in 10 μl reactions containing 5 μM random sequence decamers, reaction buffer (50 mM Tris pH 8.3, 75 mM KCl, 3 mM MgCl₂, 5 mM DTT), and 20 U/μl MMLV-RT.

The cDNAs were amplified in separate reactions. Each PCR™ comprised 100 nM of the tag primer and one of the target-specific primers (Table 3), 100 μM dGTP, dTTP, dCTP, 0.5 μM dATP and 0.5 μM [-32 P]-dATP, reaction buffer (75 mM KCl, 50 mM Tris pH 9.0, 2 mM MgCl₂), and 2.5 units SuperTaq polymerase.

PCR™ amplification was conducted by thirty cycles the following profile: 94° C., 30 seconds, 55° C., 30 seconds, 72° C., 60 seconds. At the end of the final amplification cycle, a 5 minute, 72° C. soak was used for annealing and primer extension.

### Example 9

### Quantitative Analysis of Co-Amplified Target Sequences Via Array Analysis

The amplified nucleic acids from Example 7 were prepared for array analysis by heat denaturing in 100 μl of 10 mM EDTA. The cDNA was added to the array described in Example 2 that had been prehybridized for 30 minutes at 42° C. in hybridization buffer (2.5×SSC, 7% SDS, 200 μg/ml yeast RNA). The array and cDNA were incubated overnight at 42° C. in hybridization buffer, then washed 2×30″ at 42° C. in 2×SSC/0.5% SDS and 2×30″ at 60° C. in 0.5×SSC/0.5% SDS. Autoradiography was then used to quantify the signal from each differentiation domain-specific spot.

TABLE 4

| | Copies in | Copies in |
|---|---|---|
| Target | Population #1 | Population #2 |
| IL-3 | 10¹⁰ | 10¹¹ |
| IL-4 | 10⁹ | 10⁹ |
| IL-5 | 10⁸ | 10⁷ |
| IL-7 | 10⁷ | 10⁶ |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Beasley et al, “Statistical Refinement of Primer Design Parameters,” In PCR Applications, Innis, Gelfand, Sninsky (Eds.), pp. 55-72, 1999.

Compton J. “Nucleic acid sequence-based amplification,” Nature. 7;350(6313):91-92, 1991.

Egholm et al., “PNA hybridizes to complementary oligonucleotides obeying the Watson-Crick hydrogen-bonding rules,” Nature, 365(6446):566-568, 1993.

European Patent 266,032

European Patent 329 822

European Patent 98302726

Froehler et al., “Synthesis of DNA via deoxynucleoside H-phosphonate intermediates,” Nucleic Acids Res. 14(13):5399-5407, 1986.

GB Application No. 2 202 328

Guatelli et al., “Isothermal, in vitro amplification of nucleic acids by a multienzyme reaction modeled after retroviral replication,” Proc Natl Acad Sci USA. 87(5):1874-1878, 1990.

Ikeda et al., “Selection and characterization of a mutant T7 RNA polymerase that recognizes an expanded range of T7 promoter-like sequences,” Biochemistry 32, 9115-9124, 1993.

Innis et al., “DNA sequencing with Thermus aquaticus DNA polymerase and direct sequencing of polymerase chain reaction-amplified DNA,” Proc Natl Acad Sci USA. 85(24):9436-9440, 1988.

Kato, K. “Adaptor-tagged competitive-PCR: A novel method for measuring relative gene expression,” Nuc Acids Res 25:4694-4696, 1997.

Kornberg, A. and T. A. Baker, DNA Replication, Second Edition, 1992, New York, W. H. Freeman and Company.

Kwoh et al., “Transcription-based amplification system and detection of amplified human immunodeficiency virus type 1 with a bead-based sandwich hybridization format, Proc Natl Acad Sci USA. 86(4):1173-1177, 1989.

Lizardi et al., “Mutation Detection and Single Molecule Counting Using Isothermal Rolling Circle Amplification,” Nat. Genetics 19, 225-232, 1998.

Matoba, et al., Gene 241:125-131, 2000.

PCT Application No. PCT/US89/01025

PCT Application No. WO 88/10315

PCT Application No. WO 90/07641

PCT/EP/01219

Sambrook et al., In: Molecular Cloning: A Laboratory Manual, Vol. 1, Cold Spring Harbor Laboratory Press, Cold Spring Harbor, N.Y., Ch. 7,7,19-17.29, 1989.

U.S. Pat. No. 4,659,774

U.S. Pat. No. 4,683,195

U.S. Pat. No. 4,683,202

U.S. Pat. No. 4,800,159

U.S. Pat. No. 4,816,571

U.S. Pat. No. 4,959,463

U.S. Pat. No. 5,104,792

U.S. Pat. No. 5,141,813

U.S. Pat. No. 5,210,015

U.S. Pat. No. 5,213,961

U.S. Pat. No. 5,214,136

U.S. Pat. No. 5,223,618

U.S. Pat. No. 5,262,311

U.S. Pat. No. 5,264,566

U.S. Pat. No. 5,279,721

U.S. Pat. No. 5,340,728

U.S. Pat. No. 5,378,825

U.S. Pat. No. 5,428,148

U.S. Pat. No. 5,446,137

U.S. Pat. No. 5,466,786

U.S. Pat. No. 5,470,967

U.S. Pat. No. 5,487,985

U.S. Pat. No. 5,487,993

U.S. Pat. No. 5,539,082

U.S. Pat. No. 5,545,522

U.S. Pat. No. 5,554,744

U.S. Pat. No. 5,574,146

U.S. Pat. No. 5,580,726

U.S. Pat. No. 5,599,672

U.S. Pat. No. 5,602,240

U.S. Pat. No. 5,602,244

U.S. Pat. No. 5,610,289

U.S. Pat. No. 5,614,617

U.S. Pat. No. 5,623,070

U.S. Pat. No. 5,632,957

U.S. Pat. No. 5,645,897

U.S. Pat. No. 5,652,099

U.S. Pat. No. 5,665,547

U.S. Pat. No. 5,670,663

U.S. Pat. No. 5,672,697

U.S. Pat. No. 5,681,947

U.S. Pat. No. 5,695,937

U.S. Pat. No. 5,700,922

U.S. Pat. No. 5,705,629

U.S. Pat. No. 5,708,154

U.S. Pat. No. 5,712,126

U.S. Pat. No. 5,714,331

U.S. Pat. No. 5,714,606

U.S. Pat. No. 5,719,262

U.S. Pat. No. 5,736,336

U.S. Pat. No. 5,763,167

U.S. Pat. No. 5,766,855

U.S. Pat. No. 5,773,571

U.S. Pat. No. 5,777,092

U.S. Pat. No. 5,786,461

U.S. Pat. No. 5,792,847

U.S. Pat. No. 5,834,255

U.S. Pat. No. 5,837,501

U.S. Pat. No. 5,840,873

U.S. Pat. No. 5,843,640

U.S. Pat. No. 5,843,650

U.S. Pat. No. 5,843,651

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,487

U.S. Pat. No. 5,849,497

U.S. Pat. No. 5,849,546

U.S. Pat. No. 5,849,547

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,859,221

U.S. Pat. No. 5,861,244

U.S. Pat. No. 5,861,245

U.S. Pat. No. 5,863,732

U.S. Pat. No. 5,863,753

U.S. Pat. No. 5,866,130

U.S. Pat. No. 5,866,331

U.S. Pat. No. 5,866,366

U.S. Pat. No. 5,871,697

U.S. Pat. No. 5,872,232

U.S. Pat. No. 5,882,864

U.S. Pat. No. 5,886,165

U.S. Pat. No. 5,891,625

U.S. Pat. No. 5,905,024

U.S. Pat. No. 5,910,407

U.S. Pat. No. 5,912,124

U.S. Pat. No. 5,912,145

U.S. Pat. No. 5,916,776

U.S. Pat. No. 5,919,630

U.S. Pat. No. 5,922,574

U.S. Pat. No. 5,925,517

U.S. Pat. No. 5,928,862

U.S. Pat. No. 5,928,869

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,825

U.S. Pat. No. 5,939,291

U.S. Pat. No. 5,942,391

U.S. Pat. No. 5,955,028

U.S. Pat. No. 5,962,271

U.S. Pat. No. 5,981,180

U.S. Pat. No. 6,010,850

U.S. Pat. No. 6,037,130

U.S. Pat. No. 6,057,134

WO 92/20702

Zhang et al., “Amplification of Target-Specific, Ligation-Dependent Circular Probe,” Gene 211, 277-285, 1998.

1. A method of comparing one or more nucleic acid targets within two or more samples, comprising:
a) preparing a sample mixture by a process comprising obtaining at least a first sample and a second sample, each potentially having at least a first nucleic acid target and mixing the first nucleic acid sample and the second nucleic acid sample to create a sample mixture;
b) isolating at least a first target fraction of the sample mixture;
c) performing at least a first amplification reaction on the first target fraction, wherein the amplification reaction produces at least a first amplified nucleic acid, if the first nucleic acid target is present in the first sample, and at least a second amplified nucleic acid, if the first nucleic acid target is present in the second sample;
d) differentiating the first amplified nucleic acid in the first target fraction, if any, from the second amplified nucleic acid in the first target fraction, if any; and
e) comparing abundance of the first nucleic acid target of said first sample to abundance of the first nucleic acid target of said second sample.

2. The method of claim 1, wherein the first nucleic acid target is present in the first sample.

3. The method of claim 2, wherein the first nucleic acid target is present in the second sample.

4. The method of claim 1, further defined as comprising performing at least a first amplification reaction on the first target fraction using at least a first target-specific primer, wherein the amplification reaction produces at least a first amplified nucleic acid, if the first nucleic acid target is present in the first sample, and at least a second amplified nucleic acid, if the first nucleic acid target is present in the second sample.

5. The method of claim 4, further defined as comprising isolating at least a second target fraction from the sample mixture and performing a second amplification reaction on the the second target fraction using a second target-specific primer that is specific for a second target that may be present in the first sample and/or the second sample.

6. The method of claim 1, wherein:
a) preparing the sample mixture is further defined as comprising:
preparing at least a first tagged nucleic acid sample by appending at least a first nucleic acid tag comprising a first amplification domain and a first differentiation domain to the first nucleic acid target of the first sample, if the first nucleic acid target is present in the first sample;
preparing at least a second tagged nucleic acid sample by appending at least a second nucleic acid tag comprising a second amplification domain and a second differentiation domain to the first nucleic acid target of the second sample, if the first nucleic acid target is present in the second sample, wherein the second differentiation domain is different from the first differentiation domain; and
mixing the first tagged nucleic acid sample and the second tagged nucleic acid sample to create the sample mixture; and
b) performing at least a first amplification reaction on the first target fraction is further defined as producing at least a first amplified nucleic acid comprising the first differentiation domain and a segment of the first nucleic acid target, if the first nucleic acid target is present in the first sample, and at least a second amplified nucleic acid comprising the second differentiation domain and a segment of the first nucleic acid target, if the first nucleic acid target is present in the second sample.

7. The method of claim 6, wherein the differentiation domain of the first tag and the second tag is appended between the first nucleic acid target sequence and the amplification domain.

8. The method of claim 6, wherein said nucleic acid target is one target of a plurality of nucleic acid targets within the samples.

9. The method of claim 6, wherein said first and second samples are two samples of a plurality of samples.

10. The method of claim 9, wherein the first and second tag are two tags of a plurality of tags.

11. The method of claim 6, wherein the amplification domain of the first nucleic acid tag and the second nucleic acid tag comprises a primer binding domain.

12. The method of claim 6, wherein the amplification domain of the first nucleic acid tag and the second nucleic acid tag comprises a transcription domain.

13. The method of claim 6, wherein the amplification domains of the first and second nucleic acid tags are functionally equivalent.

14. The method of claim 6, wherein the amplification domains of the first and second nucleic acid tags are identical.

15. The method of claim 6, wherein the differentiation domain of the first nucleic acid tag and the second nucleic acid tag comprise at least a size differentiation domain, an affinity domain, or a unique sequence domain.

16. The method of claim 6, wherein said first target fraction is one of a plurality of target fractions.

17. The method of claim 6, wherein the first target fraction is isolated by binding a ligand to at least a segment of the first nucleic acid target.

18. The method of claim 17, wherein the ligand is a nucleic acid, protein, or other molecule with an affinity for certain nucleic acids.

19. The method of claim 18, wherein the ligand is a nucleic acid complementary to at least a segment of the first nucleic acid target.

20. The method of claim 19, wherein the first complementary nucleic acid is used to separate the first target nucleic acid from at least one other nucleic acid or molecule.

21. The method of claim 20, wherein the target fraction is subsequently removed from the first complementary nucleic acid.

22. The method of claim 19, wherein the first complementary nucleic acid is one of a plurality of complementary nucleic acids, and each complementary nucleic acid is complementary to one of a plurality of nucleic acid targets.

23. The method of claim 19, wherein the first complementary nucleic acid is bound to a solid support.

24. The method of claim 23, wherein the first complementary nucleic acid is one of a plurality of complementary acids bound to an array, and each of the complementary nucleic acids is complementary to one of a plurality of nucleic acid targets.

25. The method of claim 23, wherein the solid support is one of a plurality of solid supports.

26. The method of claim 23, wherein the solid support is an array, a microtiter well, a chip, a bead or a combination thereof.

27. The method of claim 6, wherein differentiating comprises binding the first amplified nucleic acid to a ligand specific to at least a segment of the first differentiation domain or binding the second amplified nucleic acid to at least a segment of the second differentiation domain.

28. The method of claim 6, wherein said differentiation domain of the first nucleic acid tag comprises a first affinity domain and the second nucleic acid tag comprises a second affinity domain that is distinct from the first affinity domain.

29. The method of claim 28, wherein differentiating comprises binding at least a segment of the first affinity domain to a first affinity domain specific ligand and/or binding at least a segment of the second affinity domain to a second affinity domain specific ligand.

30. The method of claim 27, wherein the first and second affinity domain specific ligands are two of a plurality of ligands.

31. The method of claim 29, wherein at least one of the first or the second affinity domain specific ligands is bound to at least one solid support.

32. The method of claim 31, wherein the solid support is one of a plurality of solid supports.

33. The method of claim 31, wherein the solid support is an array, a microtiter well, a glass surface, a chip, a bead or a combination thereof.

34. The method of claim 29, wherein at least one of the first or the second affinity domain specific ligands is labeled.

35. The method of claim 29, wherein the binding of the first affinity domain specific ligand to the first affinity domain results in a detectable signal and/or the binding of the second affinity domain specific ligand to the second affinity domain results in a detectable signal.

36. The method of claim 35, wherein the binding of the first affinity domain specific ligand to the first affinity domain results in a first detectable signal and the binding of the second affinity domain specific ligand to the second affinity domain results in a second detectable signal.

37. The method of claim 36, wherein the first detectable signal is distinguishable from the second detectable signal.

38. The method of claim 6, wherein the differentiation domain of the first nucleic acid tag comprises a first sequence domain and the differentiation domain of the second nucleic acid tag comprises a second sequence domain that is distinguishable from the first sequence domain.

39. The method of claim 6, wherein differentiating comprises sequencing the first amplified nucleic acid and the second amplified nucleic acid.

40. The method of claim 39, wherein the amplified nucleic acid is cloned prior to sequencing.

41. The method of claim 6, wherein the differentiation domain is a unique size domain.

42. The method of claim 41, wherein differentiation comprises distinguishing amplified products by size.

43. The method of claim 6, wherein the first nucleic acid tag or the second nucleic acid tag further comprises at least one additional domain.

44. The method of claim 43, wherein said additional domain is a restriction enzyme domain, a secondary amplification domain, a sequencing primer binding site, a labeling domain or a combination thereof.

45. The method of claim 43, wherein said additional domain comprises one or more restriction enzyme domains.

46. The method of claim 43, wherein said additional domain comprises a labeling domain.

47. The method of claim 46, wherein the labeling domain is a transcription promoter.

48. The method of claim 46, wherein the labeling domain is a primer binding site.

49. A method of comparing one or more nucleic acid targets within two or more samples, comprising:
a) obtaining at least a first sample and a second sample, each potentially having at least a first nucleic acid target;
b) preparing at least a first tagged nucleic acid sample by appending at least a first nucleic acid tag comprising a first amplification domain and a first differentiation domain to the first nucleic acid target of the first sample, if the first nucleic acid target is present in the first sample;
c) preparing at least a second tagged nucleic acid sample by appending at least a second nucleic acid tag comprising a second amplification domain and a second differentiation domain to the first nucleic acid target of the second sample, if the first nucleic acid target is present in the second sample;
d) mixing the first tagged nucleic acid sample and the second tagged nucleic acid sample to create a sample mixture;
e) isolating at least a first target fraction of the sample mixture;
f) performing at least a first amplification reaction on the first target fraction, wherein the amplification reaction produces at least a first amplified nucleic acid comprising the first differentiation domain and a segment of the first nucleic acid target, if the first nucleic acid target is present in the first sample, and at least a second amplified nucleic acid comprising the second differentiation domain and a segment of the first nucleic acid target, if the first nucleic acid target is present in the second sample;
g) differentiating the first amplified nucleic acid in the first target fraction, if any, from the second amplified nucleic acid in the first target fraction, if any; and
h) comparing the first nucleic acid target of said first sample to the nucleic acid target of said second sample.

50. The method of claim 49, wherein said nucleic acid target is one target of a plurality of nucleic acid targets within the samples.

51. The method of claim 49, wherein said first and second samples are two samples of a plurality of samples.

52. The method of claim 49, wherein the amplification domains of the first and second nucleic acid tags are identical.

53. The method of claim 49, wherein the differentiation domain of the first nucleic acid tag and the second nucleic acid tag comprise at least a size differentiation domain, an affinity domain, or a unique sequence domain.

54. The method of claim 49, wherein the first target fraction is isolated by binding a ligand to at least a segment of the first nucleic acid target.

55. The method of claim 54, wherein the ligand is a first nucleic acid complementary to at least a segment of the first nucleic acid target.

56. The method of claim 54, wherein the first complementary nucleic acid is one of a plurality of complementary acids bound to an array, and each of the complementary nucleic acids is complementary to one of a plurality of nucleic acid targets.

57. The method of claim 49, wherein differentiating comprises binding the first amplified nucleic acid to a ligand specific to at least a segment of the first differentiation domain or binding the second amplified nucleic acid to at least a segment of the second differentiation domain.

58. The method of claim 49, wherein said differentiation domain of the first nucleic acid tag comprises a first affinity domain and the second nucleic acid tag comprises a second affinity domain that is distinct from the first affinity domain.

59. The method of claim 58, wherein differentiating comprises binding at least a segment of the first affinity domain to a first affinity domain specific ligand and/or binding at least a segment of the second affinity domain to a second affinity domain specific ligand.

60. The method of claim 59, wherein at least one of the first or the second affinity domain specific ligands is bound to at least one solid support.

61. The method of claim 59, wherein the binding of the first affinity domain specific ligand to the first affinity domain results in a first detectable signal and the binding of the second affinity domain specific ligand to the second affinity domain results in a second detectable signal.

62. The method of claim 61, wherein the first detectable signal is distinguishable from the second detectable signal.

63. A method of comparing one or more nucleic acid targets within two or more samples comprising
a) obtaining at least a first sample and a second sample, each potentially having at least a first nucleic acid target;
b) preparing at least a first tagged nucleic acid sample by appending at least a first nucleic acid tag comprising a first amplification domain and a first differentiation domain to the first nucleic acid target of the first sample, if the first nucleic acid target is present in the first sample, wherein the first differentiation domain comprises a first affinity domain;
c) preparing at least a second tagged nucleic acid sample by appending at least a second nucleic acid tag comprising a second amplification domain and a second differentiation domain to the first nucleic acid target of the second sample, if the first nucleic acid target is present in the second sample, wherein the first differentiation domain comprises a second affinity domain that is distinct from the first differentiation domain;
d) mixing the first tagged nucleic acid sample and the second tagged nucleic acid sample to create a sample mixture;
e) isolating at least a first target fraction of the sample mixture;
f) performing at least a first amplification reaction on the first target fraction, wherein the amplification reaction produces at least a first amplified nucleic acid comprising the first affinity domain and a segment of the first nucleic acid target, if the first nucleic acid target is present in the first sample, and at least a second amplified nucleic acid comprising the second affinity domain and a segment of the first nucleic acid target, if the first nucleic acid target is present in the second sample;
g) differentiating the first amplified nucleic acid in the first target fraction, if any, from the second amplified nucleic acid in the first target fraction, if any, by binding the first affinity domain to a first ligand and the second affinity domain to a second ligand; and
h) comparing the first nucleic acid target of said first sample to the nucleic acid target of said second sample.
